(19) United States
(12) Patent Application Publication
(10) Pub. No.: US 2026/0034121 A1
(43) Pub. Date: Feb. 5, 2026

(54) COMPOSITIONS AND METHODS FOR ALPHA-SYNUCLEIN FIBRIL GROWTH INHIBITION

(71) Applicants: Helen Hwang, St. Louis, MO (US); Paul Kotzbauer, St. Louis, MO (US); Dhruva Dhavale, St. Louis, MO (US)

(72) Inventors: Helen Hwang, St. Louis, MO (US); Paul Kotzbauer, St. Louis, MO (US); Dhruva Dhavale, St. Louis, MO (US)

(73) Assignee: Washington University, St. Louis, MO (US)

(21) Appl. No.: 19/287,405

(22) Filed: Jul. 31, 2025

Related U.S. Application Data
(60) Provisional application No. 63/678,117, filed on Aug. 1, 2024.

(51) Int. Cl. A61K 31/495; A61K 31/496; A61P 25/16; A61P 25/28

(52) U.S. Cl. CPC A61K 31/495; A61K 31/496; A61P 25/16; A61P 25/28

(57) ABSTRACT

Provided herein are compounds, compositions, and methods for inhibiting fibril growth. Compositions include at least one alpha-Synuclein (aSyn) inhibiting agent in the form of a compound including a dimethyoxyphenyl piperazine group. Methods include inhibiting aSyn fibril growth and treating a neurodegenerative disease in a subject in need thereof, including Parkinson's Disease and Lewy Body disease (LBD). Methods including administering a composition of the present disclosure. Further provided is a system for detecting aSyn fibril growth in a subject in need thereof, the system including a fluorescence screening assay of the present disclosure.

### CROSS-REFERENCE TO RELATED APPLICATIONS

This application claims priority from U.S. Provisional Application Ser. No. 63/678,117 filed 1 Aug. 2024, which is incorporated herein by reference in its entirety.

### STATEMENT OF FEDERALLY SPONSORED RESEARCH OR DEVELOPMENT

Not applicable.

### MATERIAL INCORPORATED BY REFERENCE

Not applicable.

### FIELD

The present disclosure generally relates to inhibition of alpha-Synuclein (aSyn) fibril growth.

### BACKGROUND

To date, there is no medication that can alter the disease course of Parkinson's disease. Parkinson's disease is thought to be due to alpha-synuclein fibril accumulation and growth.

Alpha-synuclein (aSyn) is a small 140 amino acid protein that regulates synaptic vesicle-mediated protein trafficking. In its native form, aSyn is a disordered soluble monomeric protein. The N-terminus forms an amphipathic alpha-helix upon interactions with membranes. In the formation of fibrils, aSyn adopts a fibrillar cross-beta sheet structure, which is considered pathologic. Misfolding of aSyn into beta-sheet amyloid fibrils coupled with accumulation is linked to the pathophysiology of Parkinson's disease (PD) and Lewy body disease (LBD), the dementia associated with PD. Furthermore, dominantly inherited mutations in the gene encoding aSyn (SNCA) have been identified in rare forms of hereditary parkinsonism that are also characterized by aSyn fibril accumulation, supporting aSyn fibril accumulation as a therapeutic target.

Asyn fibril accumulation involves an initial nucleation event, where two or more aSyn monomers come together to form a misfolded oligomeric seed or protofibril. Nucleation is followed by the growth phase or elongation phase, where the sequential addition of aSyn monomers leads to the formation of fibrils with beta-sheet structure. Previous studies have utilized several approaches to identify inhibitors for aSyn fibril nucleation and growth. Stabilizing the monomeric form of aSyn by DNA aptamers has been shown to delay fibril formation. Others have shown that some polyphenols, including (−)-epigallocatechin gallate (EGCG) can redirect monomeric a-synuclein to form off-pathway spherical oligomers and thus inhibit beta-sheet fibril formation. Dopamine and other catecholamines can similarly promote the formation of non-fibrillar oligomeric forms of aSyn. Other molecules, such as select antibiotics, pigments, glucosides, quinones, and pirimido-pyrazine also have the potential to inhibit aSyn aggregation.

Conventional in vitro screening approaches to identify inhibitors of aSyn fibril accumulation commonly utilize a non-seeded aggregation approach. One study adopted a seed amplification approach with repeated incubation and sonication to achieve exponential growth. Both types of studies typically use high monomeric aSyn concentrations (25-140 μM), continuous orbital rotation, and lengthy incubation time (16-48 hours).

Asyn fibril accumulation is typically quantified by measuring Thioflavin T (ThioT) fluorescence, either continuously or at an endpoint after a 24-36 hour incubation period. ThioT is a fluorescent dye that is weakly fluorescent in the presence of monomeric aSyn but undergoes a substantial increase in fluorescence intensity by several orders of magnitude upon binding to amyloid fibrils, including the beta sheets of aSyn fibrils. A fluorescence-based fibril growth assay that uses fluorescein arsenical hairpin binder (FlAsH) to detect the association of two or more aSyn monomers with bicysteine tags has been developed. The FlAsH assay relies on quiescent incubation of monomeric aSyn with a bicysteine (C2) tag and wildtype aSyn fibril seeds with no C2 tag and can be used to study linear rates of fibril growth when either soluble oligomers or sonicated fibril seeds are combined with monomeric aSyn. Furthermore, the incubation time is short (3 hours), during which the rate of fibril growth is constant. Since wild-type (WT) aSyn fibril seeds produce no FLASH fluorescence, the fluorescence signal is specific to fibril elongation with C2-aSyn. It was previously observed similar growth kinetics with C2-aSyn monomer and WT-aSyn monomer, using ThioT as a readout, suggesting that addition of a bicysteine tag on the N terminus of aSyn does not interfere with fibril growth. Previous experiments comparing ThioT and FlAsH show that assays utilizing FlAsH fluorescence measurements have a 4 to 60-fold higher Z′-factor, a measure of the dynamic range and separation between signal and controls, as well as a 10-fold increase in signal/background ratio compared to ThioT. Given the higher sensitivity of FlAsH dye compared to ThioT fluorescence, this approach is able to detect small changes in fibril growth over shorter periods of time (3 hours), providing several advantages as a screening assay.

### BRIEF DESCRIPTION OF THE DISCLOSURE

Among the various aspects of the present disclosure is the provision of compositions and methods thereof for inhibition and prevention of alpha-Synuclein (aSyn) fibril growth, including for treating Parkinson's disease. Compounds and compositions thereof described herein inhibit the growth of fibrils amplified from Parkinson's Disease or Lewy Body disease (LBD) brain tissue, in some embodiments by binding to the elongating ends of fibrils, potentially “capping” them.

In accordance with an aspect of the present disclosure, a compound is provided as described herein.

In accordance with another aspect of the present disclosure, a composition is provided. The composition comprising at least one compound described herein.

In accordance with a further aspect of the present disclosure, a method of inhibiting alpha-synuclein (aSyn) fibril growth in a subject in need thereof is provided. The method comprising: administering a composition comprising at least one compound described herein.

In accordance with an additional aspect of the present disclosure, a method of treating Parkinson's Disease in a subject in need thereof is provided. The method comprising: administering a composition comprising at least one compound described herein.

In accordance with yet another aspect of the present disclosure, a method of treating Lewy Body disease (LBD) in a subject in need thereof is provided. The method comprising: administering a composition comprising at least one compound described herein.

In accordance with yet a further aspect of the present disclosure, a system for detecting alpha-synuclein (aSyn) fibril growth in a subject in need thereof is provided. The the system comprising: a fluorescence screening assay described herein.

In one aspect of the present disclosure, a composition comprising at least one alpha-Synuclein (aSyn) inhibiting agent is provided. The at least one aSyn inhibiting agent is a compound comprising a dimethyoxyphenyl piperazine group according to

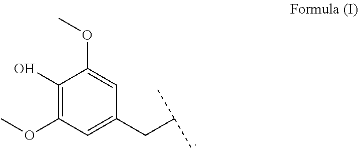

In some embodiments, the compound comprising a dimethyoxyphenyl piperazine group is a compound according to

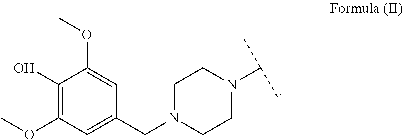

In some embodiments, the neurodegenerative disease is Parkinson's Disease, the neurodegenerative disease is Lewy Body disease (LBD), the inhibiting aSyn fibril growth further comprises preventing accumulation of aSyn fibrils in the subject. and/or the at least one aSyn inhibiting agent is selected from Compounds 5 and 8-24.

In another aspect of the present disclosure, a method of inhibiting alpha-synuclein (aSyn) fibril growth in a subject in need thereof. The method comprising: administering to the subject a composition comprising at least one alpha-Synuclein (aSyn) inhibiting agent, wherein the at least one aSyn inhibiting agent is a compound comprising a dimethyoxyphenyl piperazine group according to % Formula (1).

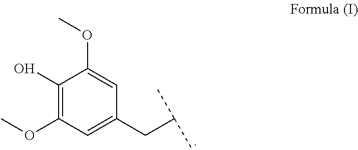

In some embodiments, the compound comprising a dimethyoxyphenyl piperazine group is a compound according to

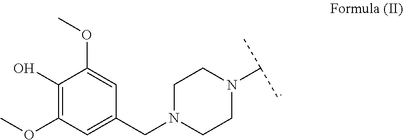

In some embodiments, the neurodegenerative disease is Parkinson's Disease, the neurodegenerative disease is Lewy Body disease (LBD), the inhibiting aSyn fibril growth further comprises preventing accumulation of aSyn fibrils in the subject. and/or the at least one aSyn inhibiting agent is selected from Compounds 5 and 8-24.

In a further aspect of the present disclosure, a method of treating a neurodegenerative disease in a subject in need thereof is provided. The method comprising: administering to the subject a composition comprising at least one alpha-Synuclein (aSyn) inhibiting agent, wherein the at least one aSyn inhibiting agent is a compound comprising a dimethyoxyphenyl piperazine group according to

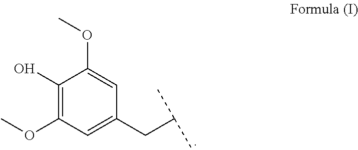

In some embodiments, the compound comprising a dimethyoxyphenyl piperazine group is a compound according to

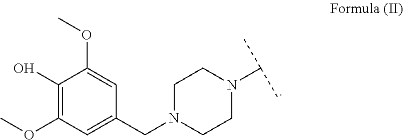

In some embodiments, the neurodegenerative disease is Parkinson's Disease, the neurodegenerative disease is Lewy Body disease (LBD), and/or the at least one aSyn inhibiting agent is selected from Compounds 5 and 8-24.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1 is a schematic of fibril growth inhibition with a proposed mechanism of inhibition of aSyn fibril growth by a small molecule inhibitor (pink) binding to the end surface of fibrils.

FIG. 2A is a representative dose-response curve for EGCG in an optimized fibril growth assay based on FlAsH fluorescence. Note the increased fluorescence signal for monomer plus EGCG at 30 μM concentration, limiting the measurement of fibrils at this concentration.

FIG. 2B is a representative dose-response curve for 1 in the optimized fibril growth assay. The fluorescence signal is low in the presence of monomer only and does not increase significantly with increasing compound concentrations.

FIG. 2C is the chemical structure of 1 with shaded areas indicating regions where modifications were tested for structure-activity relationship (SAR) studies.

FIG. 2D is the chemical structure of 10 with shading that indicates the region that was modified for the third set of structure-activity relationship studies.

FIG. 2E is a representative dose-response curve for 10 in the optimized fibril growth assay. The fluorescence signal is low in the presence of monomer only and does not increase significantly with increasing compound concentrations. Data points represent mean±standard deviation (std) (triplicates; n=3 independent experiments).

FIG. 3A is a pair of dose-response curves for 8 (top) and 10 (bottom) using a conformational-specific immunoassay to measure inhibition of fibril growth.

FIG. 3B is a pair of corresponding dose-response curves for 8 (top) and 10 (bottom) using a FlAsH fluorescence assay to measure inhibition of fibril growth; IC₅₀ is mean±std from two independent experiments. Note that the immunoassay detects both seeds and new fibril growth while the FlAsH assay detects only new fibril growth, accounting for differences in the apparent maximum reduction in signal by compounds.

FIG. 4A is a graph of the measurement of fibril growth by ThioT fluorescence in the presence of 8 and 10 at 30 μM and 100 μM respectively, after serial centrifugation and washing. There is significant fibril growth inhibition seen at both concentrations of 8 and 10 (t-test, p<0.01). Graphs show data points (n=3) with mean (bars) and std (error bars). Similar results were observed in three independent experiments.

FIG. 4B is a plot of fluorescence readings over time for a kinetic seeded assay measuring ThioT fluorescence in the absence or presence of 8 and 10 at 30 μM and 100 μM respectively with continuous orbital shaking for 48 hours. The line represents smoothed data averaging over 3 neighboring data points.

FIG. 4C is graph of lag time measurements graphed for each condition in the seeded kinetic assay, defined by the time point at which fluorescence intensity increases by 20% above the baseline. There is significant increase in lag time seen at both concentrations of 8 and 10 (t-test, p<0.05). Graphs show mean (data points) plus std (error bars). Similar results were observed from three independent experiments.

FIG. 5A is a dose-response curves of 8 (see FIG. 4A) in FlAsH fibril growth assays using fibrils amplified from LBD brain tissue. Graphs show mean (data points) plus std (error bars). Similar results were observed from two independent experiments.

FIG. 5B is a dose-response curves of 10 (see FIG. 4B) in FlAsH fibril growth assays using fibrils amplified from LBD brain tissue. Graphs show mean (data points) plus std (error bars). Similar results were observed from two independent experiments.

FIG. 6A is a schematic of molecular docking for compound 10 on the surface of the elongating ends of fibrils, where they could inhibit binding of monomeric aSyn required for elongation. Example docking sites of 10 on LBD aSyn fibrils (8a9I).

FIG. 6B is another schematic of molecular docking for compound 10 on the surface of the elongating ends of fibrils, where they could inhibit binding of monomeric aSyn required for elongation. Example docking sites of 10 on LBD aSyn fibrils (8a9I).

FIG. 6C is a schematic of molecular docking for compound 8 on the surface of the elongating ends of fibrils, where they could inhibit binding of monomeric aSyn required for elongation. Example docking sites of 8 on LBD aSyn fibrils (8a9I).

FIG. 6D is a schematic of molecular docking for compound 8 on the surface of the elongating ends of fibrils, where they could inhibit binding of monomeric aSyn required for elongation. Example docking sites of 8 on LBD aSyn fibrils (8a9I).

FIG. 7A is a graph showing FlAsH fluorescence intensity increases in a linear fashion with increasing fibril concentrations and 3 μM aSyn monomer.

FIG. 7B is a graph showing that fibril growth also increases in a linear fashion with low monomer concentrations (up to 6 μM) with 1 μM aSyn fibril.

FIG. 7C is a graph of fibril growth as measured by FlAsH fluorescence that increases in a linear fashion at 3 and 6 hours.

FIG. 8A is a table of summary of results of initial SAR studies (SAR1) that shows minimal aSyn fibril growth inhibition with substitutions in the benzyl group (left-side) of 1. IC50 is mean±std. Max inhibition is calculated as (Intensity100 μM−Intensity0 μM)/intensity0 μM×100%. N=2 independent experiments.

FIG. 8B is a table of results of SAR2 studies with substitutions to the right-side of 1. IC50 is mean±std from 2 different independent experiments. Max inhibition is calculated as (Intensity100 μM−Intensity0 μM)/intensity0 μM×100%.

FIG. 9(A-B) is a table of results of SAR3 studies. IC50 is mean±std. Max inhibition is calculated as (Intensity100 μM−Intensity0 μM)/intensity0 μM×100%. N=2 independent experiments. FIG. 9A shows compounds 14-22. FIG. 9B shows compounds 23-30.

FIG. 10 is a graph showing that compounds 8 and 10 do not disassemble fibrils. FLASH fluorescence measurements of C2-aSyn fibrils in the absence of aSyn monomer after a 3-hour incubation in the absence or presence of compounds 8 and 10 reveal no change in fluorescence. Data points represent mean±std of triplicates from two independent experiments.

FIG. 11 is a set of negative stain TEM images that indicate that there is no change in fibril morphology when fibrils are incubated with compound 8 or 10. TEM images of sonicated in vitro aSyn fibrils incubated for 3 hours with monomer plus compound 8, 10 or no compound. Fibril morphology for the in vitro aSyn fibrils is characterized by two protofilaments of about 9 to 10 nm diameter each for intact fibrils, which are fragmented by sonication at the beginning of the growth period. Fibrils appear similar with respect to diameter and overall morphology in the presence or absence of compound, with short fragments of two protofilament and single protofilament fibrils in close association on the grid.

FIG. 12 is a graph of relative sensitivity of the immunoassay for fibrils and monomers. IC50 is mean±std. This assay is approximately 300-fold more sensitive for detecting fibrils than monomers.

FIG. 13A is a pair of graphs of fluorescence intensity of ThioT plus fibrils without compounds (fibrils only) and ThioT plus fibrils in the presence of 100 μM of 8 and 10, incubated in the absence of monomer. Reduced ThioT fluorescence signal is observed in the presence of 8 and 10, (t-test, p<0.05).

FIG. 13B is a graph showing that, after serial wash and centrifugation to remove compounds 8 and 10, there is no significant difference in ThioT fluorescence between the test conditions (t-test, p<0.05). Measurements were performed after incubation with DMSO buffer or 100 μM compounds. Data points represent mean±std of triplicates from three independent experiments.

FIG. 14A is a graph of a competitive binding assay in which fibrils were incubated with 3 μM ThioT and increasing concentrations of compound 8, using in vitro aSyn fibrils as binding substrate. The results show modest reductions in ThioT fluorescence in the presence of a high concentration (100 μM) of compound 8 for both nonsonicated and sonicated fibrils, indicating no difference when the number of fibril ends is increased by sonication.

FIG. 14B is a graph of a competitive binding assay in which fibrils were incubated with 3 μM ThioT and increasing concentrations of compound 8, using sonicated in vitro aSyn fibrils as binding substrate. The results show modest reductions in ThioT fluorescence in the presence of a high concentration (100 μM) of compound 8 for both nonsonicated and sonicated fibrils, indicating no difference when the number of fibril ends is increased by sonication.

FIG. 14C is a graph of a competitive binding assay with increasing concentrations of compound 10 for in vitro aSyn fibrils. Data points represent mean±std of triplicates. Similar results were observed in two independent experiments.

FIG. 14D is a graph of a competitive binding assay with increasing concentrations of compound 10 for sonicated in vitro aSyn fibrils. Data points represent mean±std of triplicates. Similar results were observed in two independent experiments.

FIG. 15 is a graph of initial slopes measured for the rise in fluorescence intensity following the range of lag periods observed for fibrils incubated with ThioT in the presence or absence of compounds 8 and 10, in the experiment shown in FIG. 4B. Initial slopes were determined by measuring the average increase in fluorescence intensity over time during the 3-hour period following the lag time. The rate of fibril growth as measured by the slope is significantly lower in the presence of compounds 8 and 10 compared with no compound. Bar graph represent mean±std of quadruplicates from three independent experiments.

FIG. 16(A-C) shows molecular docking results for compound 8 on LBD-derived fibrils (8a9I) with calculated free energy affinity for each predicted binding site. FIG. 16A shows modes 1-3; FIG. 16B shows modes 4-6; FIG. 16C shows modes 7-9.

FIG. 17(A-C) shows molecular docking results for compound 10 on LBD-derived fibrils (8a9I) with calculated free energy affinity for each predicted binding site. FIG. 17A shows modes 1-3; FIG. 17B shows modes 4-6; FIG. 17C shows modes 7-9.

### DETAILED DESCRIPTION OF THE DISCLOSURE

As disclosed herein, structure-activity testing has resulted in a new class of molecules that are more potent inhibitors of alpha-synuclein aggregation (e.g., in Parkinson's Disease). In exemplary embodiments, the disclosed compounds inhibit or prevent a-Syn aggregation and slow down the onset of the disease phenotype (motor dysfunction) and neurodegenerative changes of Parkinson's Disease.

Accordingly, the present disclosure is directed to compounds that inhibit Alpha-Synuclein fibril growth in treating Parkinson's disease. Alpha-synuclein fibril accumulation is the defining feature of Parkinson's disease and a target for disease-modifying treatments. Compound 1 (ethyl 1-[(4-hydroxy-3,5-dimethoxyphenyl) methyl]piperidine-4-carboxylate) is a lead inhibitor of fibril growth. The analogs of 1 can be divided into classes of phenylethanoid (8) and dimethoxy phenols (10), which are more potent and have more significant maximum inhibition than the original 1. Analogs 8 and 10 also inhibit the growth of A-syn fibrils amplified from post-mortem Lewy Body disease (LBD) brain tissue, validating the disclosed in-vitro fibril growth assay. They can bind parallel to the fibril beta-sheet, potentially “capping” them, blocking the fibril end and preventing further monomer addition, thereby reducing fibril accumulation. These compounds enable development of disease-modifying therapies for neurodegenerative diseases, including but not limited to Parkinson's disease and Lewy Body Disease. In some aspects, isolating and targeting the fibril growth pathway can yield additional candidate inhibitors for drug discovery.

In some aspects, the present disclosure optimizes a fibril growth assay utilizing FlAsH fluorescence to screen and identify small molecule compounds that inhibit aSyn fibril growth. In other aspects, the method enables the discovery of new compounds by measuring aSyn fibril growth over short time periods (3 hours), using low concentrations of fibril seeds and monomer, where fibril growth is linear with respect to time.

In some aspects, an IC₅₀ value was obtained for EGCG in the FlAsH fluorescence assay that is similar to IC₅₀ values previously obtained using ThioT to measure inhibition of aSyn fibril growth. In other aspects, using the FlAsH fluorescence assay, a family of dimethoxyphenyl piperazine compounds capable of inhibiting fibril growth in-vitro were identified. In accordance with another aspect, fibril growth inhibition was further validated with two ThioT fluorescence assays and an aSyn conformation-specific immunoassay. In other aspects, the four assays produced similar results, but the FlAsH fluorescence assay detects smaller changes in fibril growth over shorter periods of time and with greater precision. This is partly due to the absence of signal and noise from fibril seeds that do not contain C2-tags.

In some aspects, the immunoassay and ThioT assays detect initial fibril seeds as well as fibril growth, and hence, are less sensitive to small amounts of fibril growth that occurs in 3 hours. In other aspects, the concentration of fibril seeds and monomer is increased, and the incubation time is extended (20 hours), as compensation mechanisms in both the immunoassay and ThioT fluorescence experiments. In some aspects, the changes in fibril concentration measured under these modified assay conditions may reflect other molecular events in addition to fibril elongation, including primary or secondar nucleation of oligomeric intermediates and conversion to fibrils. In yet other aspects, in the case of the modified kinetic assay that utilizes shaking in a plate reader and intermittent ThioT fluorescence measurements, where a lag phase followed by a rapid rise in fibril concentration is observed, a combination of nucleation events, intermittent fibril fragmentation and elongation of fibrils may best explain the changes in fibril concentration. In another aspect, the observed inhibition by compounds 8 and 10 in these additional assays indicates that these compounds can effectively inhibit fibril accumulation in a variety of assay formats.

Previous studies identified multiple locations where a single amino acid difference between monomeric aSyn and fibril seeds can substantially alter the fibril growth rate. Some amino acid residues within the beta-sheet region may be more important than others for determining the rate at which monomeric aSyn associates with the elongating end of the fibril, since some amino acid changes had no effect on growth rates. These previous studies of fibril growth indicate that elongation requires specific conformational alignments as each monomeric unit associates with the fibril end. They also suggest that interaction of a small molecule compound with only two or three amino acid residues in either the fibril end or monomeric aSyn has the potential to inhibit elongation.

In some aspects, the docking studies indicate that dimethoxyphenyl piperazine compounds may inhibit fibril growth by binding to fibril ends at the elongating surface of fibrils and inhibiting the association of additional monomers. However, it is also possible that the compounds bind to monomeric aSyn in ways that inhibit its association with fibrils or interfere with a conformational change required to associate with the fibril end. Other previously identified small molecules such as EGCG inhibit fibril growth by interacting with monomer and sequestering it through the formation of off-pathway oligomers to prevent association with elongating fibrils. An increase in FlAsH signal with incubation of monomeric aSyn with EGCG was observed, which is likely related to formation of oligomeric species. In contrast, FlAsH assays do not indicate the formation of oligomeric species when monomeric aSyn is incubated with the dimethoxyphenyl piperazine compounds.

In some aspects, potential mechanisms based on interaction with either fibrils or monomer may be difficult to distinguish through further kinetic studies with varying concentrations of monomer or fibrils. For example, increasing monomer concentration may decrease the IC₅₀ for compound inhibition by increasing the number of binding sites available on monomer or alternatively by promoting greater competition of monomer with compound at binding sites on fibril ends. Increasing fibril seed concentration may increase the number of binding sites available on fibril ends but may not change IC₅₀ if it does not reduce free monomer concentration and also may not change the IC₅₀ if compound is binding to monomer. Future studies using cryo-EM, NMR and radioligand binding assays with radiolabelled compounds of interest could provide additional information about binding sites and potential mechanisms. Further structural understanding of binding sites on fibrillar and monomeric aSyn can guide the optimization of compound structure to improve potency.

To date, there have been several pre-clinical and clinical studies focusing on small molecules that prevent aSyn fibril accumulation. EGCG, which has been shown to inhibit aSyn fibril growth, failed to show a protective effect in participants with multiple systems atrophy, another synucleinopathy (phase 3, PROMESA trial). This could be due to the limited blood-brain-barrier penetration of EGCG. Other small molecules currently in phase 2 trials include a pyrazino-pyrimidine NTP200-11 (UCB0599, NCT04658186) and herbal ethanolic extract MT101-5 (Mthera Pharma Co, NCT06175767). There are ongoing efforts to study the tolerability of small molecules of a pyrazole moiety Anle138b (MODAG GmbH, phase 1 NCT04685265) that have been shown to decrease aSyn aggregation in the presence of lipids. Furthermore, there have been promising results from preclinical studies of small molecule compound SynuClean-D in vitro and in human neuroglioma cells and with molecular tweezers (Clr01) in-vitro and in mouse models.

In some aspects, with further optimization and validation, the assays described herein may be suitable for high throughput screening of compound libraries to identify additional new leads for this PD therapeutic approach. Although the compounds identified here appear equally potent in assays with in vitro and LBD aSyn fibrils, there may be advantages to utilizing aSyn fibrils derived from postmortem LBD tissue in fibril growth assays as a primary screening approach for small molecule inhibitors. Previous studies indicate that different aSyn fibril conformers have distinct structural requirements for fibril growth, and several amino acid residues that are critical for LBD aSyn fibril growth but not for in vitro fibrils were identified. Future studies to understand how the compounds identified here and in other studies inhibit LBD aSyn fibril growth may also provide further guidance for optimizing inhibition potency in efforts to develop effective mechanism-based therapeutics targeting aSyn fibril accumulation in PD.

In some aspects, aSyn fibril growth is a process that can be targeted to prevent the progressive accumulation of aSyn fibrils in PD. In another aspect, the assay described herein provides a platform for identifying potent aSyn fibril growth inhibitors. In yet other aspects, these small molecules can be further developed as disease-modifying therapies for PD.

### Asyn Inhibiting Agent

One aspect of the present disclosure provides for targeting of aSyn fibril growth. The present disclosure provides methods of treating or preventing Parkinson's Disease based on the discovery that compounds designed and disclosed herein bind to elongated ends of fibrils amplified from Lewy Body disease (LBD) brain tissue, thus “capping” them to prevent and/or inhibit growth.

As described herein, an aSyn inhibiting agent comprises at least one compound described herein. An aSyn inhibiting agent can include one or more compounds, including at least one compound disclosed herein, such that the agent is effective to inhibit aSyn growth.

In some embodiments, inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Chemical Agent

Examples of aSyn inhibiting agents are described herein. Agents can include one or more of the compounds described herein, or pharmaceutically acceptable salts thereof, including dimethyoxyphenyl piperazine compounds of

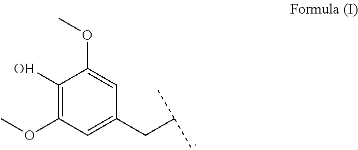

In some embodiments, aSyn inhibiting agents include compounds of

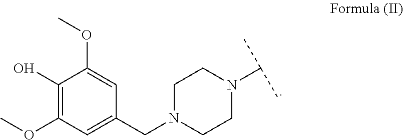

The formulas, analogs, and R groups (including those groups constituting the dashed line remainder of the aSyn inhibiting agent compounds according to Formula (I) and/or Formula (II) as embodied herein) can be optionally substituted or functionalized with one or more groups independently selected from the group consisting of hydroxyl; C₁₋₁₀alkyl hydroxyl; amine; C₁₋₁₀carboxylic acid; C₁₋₁₀-carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl,-n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O— cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH2-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Neurodegenerative Disease

The compositions and methods as described herein can be used to treat a neurodegenerative disease, disorder or condition. In exemplary embodiments, the compounds, compositions, and methods described herein are used to treat a neurodegenerative disease, disorder or condition in which inhibiting and/or preventing fibril growth is required or desired.

For example, a neurodegenerative disease, disorder or condition can be a hereditary motor and sensory neuropathy (HMSN) (e.g., Charcot Marie Tooth (CMT) disease), CMT1 (a dominantly inherited, hypertrophic, predominantly demyelinating form), CMT2 (a dominantly inherited predominantly axonal form), Dejerine-Sottas (severe form with onset in infancy), CMTX (inherited in an X-linked manner), CMT4 (includes the various demyelinating autosomal recessive forms of Charcot-Marie-Tooth disease), hereditary sensory and autonomic neuropathy type IE, hereditary sensory and autonomic neuropathy type II, hereditary sensory and autonomic neuropathy type V, HMSN types 1A and 1B (e.g., dominantly inherited hypertrophic demyelinating neuropathies), HMSN type 2 (e.g., dominantly inherited neuronal neuropathies), HMSN type 3 (e.g., hypertrophic neuropathy of infancy [Dejerine-Sottas]), HMSN type 4 (e.g., hypertrophic neuropathy [Refsum] associated with phytanic acid excess), HMSN type 5 (associated with spastic paraplegia), or HMSN type 6 (e.g., with optic atrophy).

As another example, a neurodegenerative disease, disorder or condition can be Alzheimer's disease, amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjögren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, GIn by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C. +16.6(log₁₀[Na⁺])+0.41 (fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | |
| Uncharged-polar | |
| A. Hydroxyl: | S T Y |
| B. Amides: | N Q |
| C. Sulfhydryl: | C |
| D. Borderline: | G |
| Positively Charged | |
| (Basic): | K R H |
| Negatively Charged | |
| (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, gene and/or protein expression signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of gene/protein expression/signaling by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies for various diseases are rapidly advancing.

There has recently been an improved landscape for gene therapies. For example, in the first quarter of 2019, there were 372 ongoing gene therapy clinical trials (Alliance for Regenerative Medicine, 5/9/19).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses transcribing |
| | their single-stranded genome into a double- |
| | stranded DNA copy, which can integrate into |
| | host chromosome |
| Adenoviruses (Ad) | Ad can transfect a variety of quiescent and |
| | proliferating cell types from various species |
| | and can mediate robust gene expression |
| Adeno-associated | Recombinant AAV vectors contain no viral |
| Viruses (AAV) | DNA and can carry ~4.7 kb of foreign |
| | transgenic material. They are replication |
| | defective and can replicate only while |
| | coinfecting with a helper virus |
| Non-viral vectors | |
| plasmid DNA | pDNA has many desired characteristics as a |
| (pDNA) | gene therapy vector; there are no limits on |
| | the size or genetic constitution of DNA, it is |
| | relatively inexpensive to supply, and unlike |
| | viruses, antibodies are not generated against |
| | DNA in normal individuals |
| RNAi | RNAi is a powerful tool for gene specific |
| | silencing that could be useful as an enzyme |
| | reduction therapy or means to promote read- |
| | through of a premature stop codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing Parkinson's Disease in a subject in need thereof via administration of a therapeutically effective amount of an aSyn inhibiting agent, so as to inhibit or prevent aSyn fibril growth.

Methods described herein are generally performed on a subject in need thereof. A subject in need of the therapeutic methods described herein can be a subject having, diagnosed with, suspected of having, or at risk for developing Parkinson's Disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an aSyn inhibiting agent is, for example, an amount that would cause the desired therapeutic effect in a subject while minimizing undesired side effects. In various embodiments, an effective amount of an aSyn inhibiting agent described herein can substantially inhibit, slow the progress of, or limit the development of Parkinson's Disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an aSyn inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit or prevent aSyn fibril growth.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an aSyn inhibiting agent can occur as a single event or over a time course of treatment. For example, an aSyn inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for Parkinson's Disease.

An aSyn inhibiting agent can be administered simultaneously or sequentially with another agent, such as an antibiotic, an anti-inflammatory, or another agent. For example, an aSyn inhibiting agent can be administered simultaneously with another agent, such as an antibiotic or an anti-inflammatory. Simultaneous administration can occur through administration of separate compositions, each containing one or more of an aSyn inhibiting agent, an antibiotic, an anti-inflammatory, or another agent. Simultaneous administration can occur through administration of one composition containing two or more of an aSyn inhibiting agent, an antibiotic, an anti-inflammatory, or another agent. An aSyn inhibiting agent can be administered sequentially with an antibiotic, an anti-inflammatory, or another agent. For example, an aSyn inhibiting agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et aL., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the aSyn inhibiting agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, an aSyn inhibiting agent such as a compound described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to compounds, compositions, and formulations described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Identification of Small Molecule Dimethyoxyphenyl Piperazine Inhibitors of Alpha-Synuclein Fibril Growth

Alpha-synuclein (aSyn) fibril accumulation is the defining feature of Parkinson's disease and is a target for disease-modifying treatments. One therapeutic strategy to reduce fibril accumulation is inhibition of aSyn fibril growth. In Example 1, a sensitive fluorescence-based fibril growth assay was developed to screen for small molecule inhibitors. After validating the inhibition assay using a previously identified inhibitor, epigallocatechin-3-gallate, compound 1 was identified as a lead for inhibition of fibril growth. Structure-activity relationships were analyzed with analogs of 1 to optimize inhibition potency. The results identified two dimethoxyphenyl piperazine analogs with more potent inhibition of in-vitro assembled fibrils, which were further validated with orthogonal assays including kinetic measurements of fibril concentration with Thioflavin T. These analogs also inhibited the growth of aSyn fibrils amplified from Lewy Body Disease brain tissue, further validating the inhibitor screening assay. Molecular docking studies indicate that these compounds can bind to the fibril ends, suggesting a potential capping mechanism through which these compounds inhibit the sequential association of monomeric aSyn required for fibril growth.

More specifically, the FlAsH fibril growth assay was further developed to screen small molecule compounds by using lower concentrations of monomer and fibril seeds and a short incubation period. That this fibril growth assay measures an inhibitory effect of EGCG on fibril growth as observed in previous studies with ThioT was validated and then this fibril growth assay was used to identify lead compound 1. To identify more potent inhibitors, a series of structure-activity testing experiments were performed and two analogs of 1 that were more potent inhibitors were identified. These analogs of 1 also inhibited the growth of aSyn fibrils amplified from postmortem Lewy Body disease (LBD) brain tissue.

### Results

### An In Vitro Asyn Fibril Growth Assay for Screening Small Molecule Inhibitors

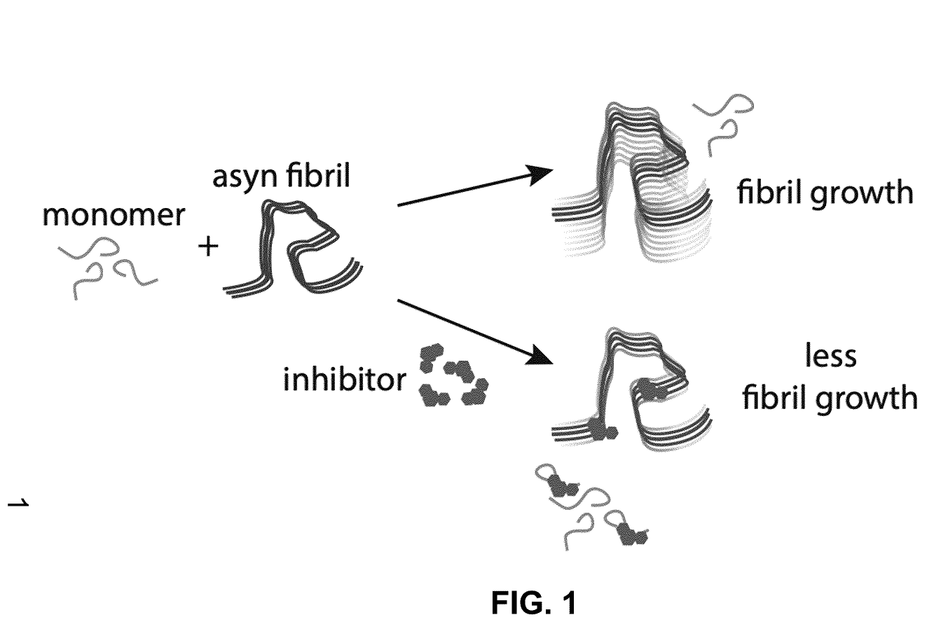

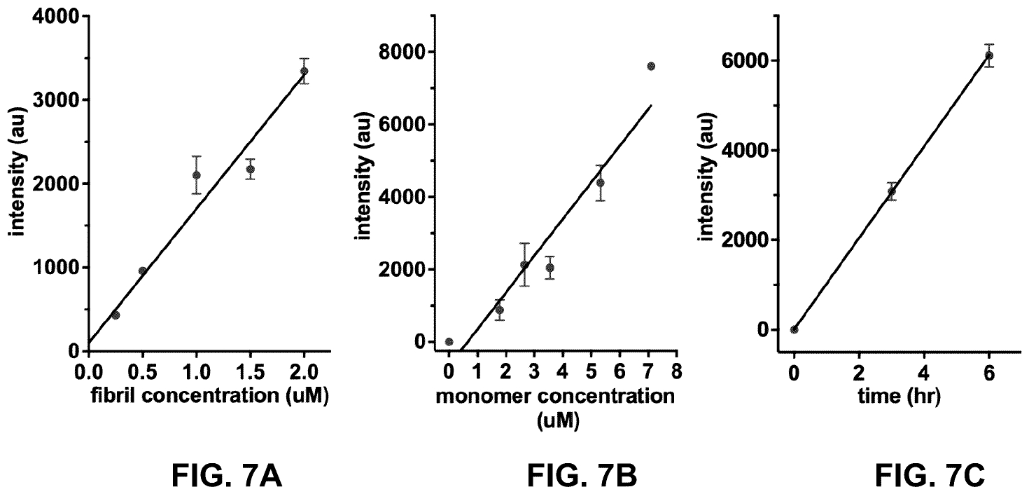

Inhibition of fibril growth by small molecule compounds is likely to be mediated primarily by binding interactions with either monomer, fibrils, or both. However, secondary nucleation may occur and contribute to fibril growth under some conditions, and compounds may affect growth through interaction with oligomeric intermediates to inhibit conversion to fibrils. In one proposed mechanism, a compound binds to the end surface of fibrils and interferes with the binding of the new monomer required to extend the seed or fibril (FIG. 1). Since binding will deplete the free concentration of the inhibitor, measured IC₅₀ values will be proportional to the concentration of the binding target (fibril seeds) and proportional to the concentration of monomer, with which the inhibitor competes for binding. Linear increases in fibril concentration were previously observed when the incubation time was varied and a linear increase in FlAsH fluorescence was observed. Thus, optimization of the FlAsH fibril growth assay was performed by decreasing the concentrations of monomer and fibril seeds and it was found that a combination of 1 μM fibril seeds, 3 μM monomer, and 3-hour incubation time provided sufficient signal to analyze dose-response curves and determine IC₅₀ values. Under these assay conditions, linear relationships between aSyn fibril concentration and FlAsH fluorescence and between aSyn monomer concentration and FlAsH fluorescence were observed (FIGS. 7A and 7B). The increase in FlAsH fluorescence intensity was linear between 3 and 6 hours (FIG. 7C), indicating sufficient monomer concentration to maintain a constant growth rate over this time period, but the rate of increase declined beyond 6 hours (data not shown). These results are supportive of the assay primarily measuring rate of fibril elongation under these conditions. In some embodiments, the assay can further be utilized to measure/quantify the elongation process.

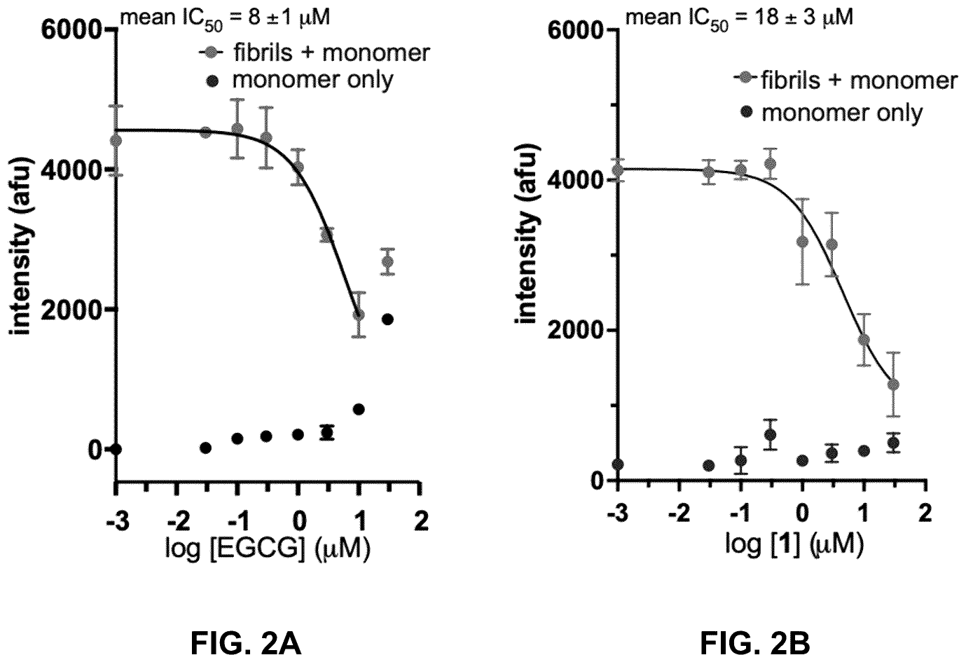

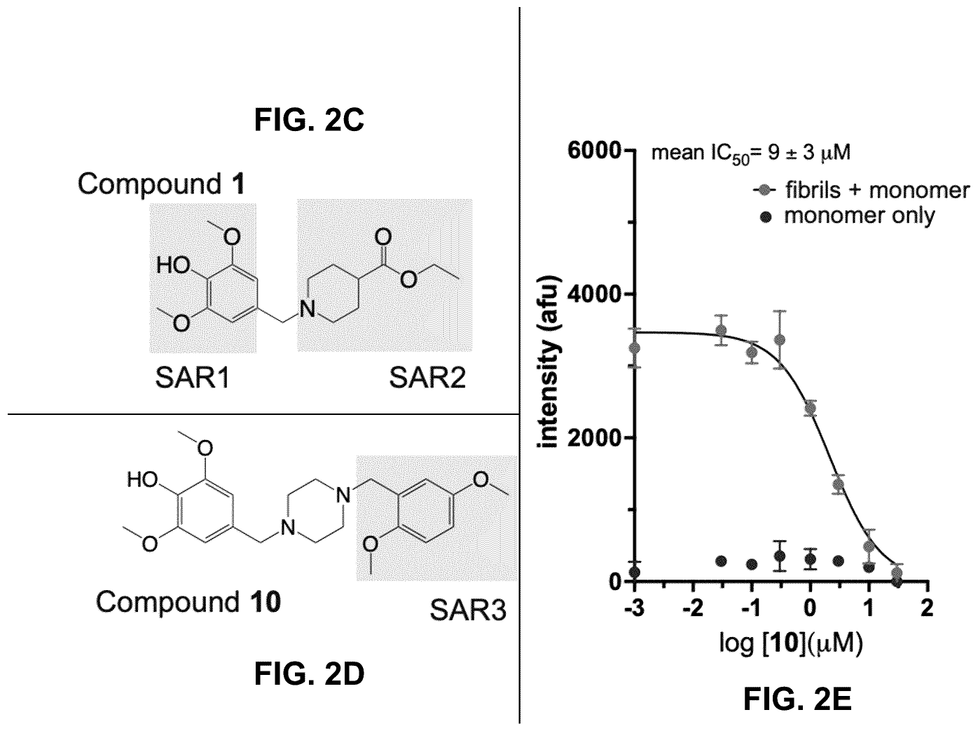

The optimized FlAsH assay was used to evaluate a previously identified fibril growth inhibitor, EGCG, and dose-dependent inhibition with an inhibition constant (IC₅₀) of 8±1 μM was observed (FIG. 2A). Interestingly, control reactions containing EGCG with no seeds showed increased fluorescence signal at high EGCG concentrations. This may correspond to the formation of off-pathway oligomers previously observed when aSyn is incubated with EGCG. A compound previously identified as a potential ligand for aSyn was then tested in molecular docking studies, ethyl 1-[(4-hydroxy-3,5-dimethoxyphenyl)methyl]piperidine-4-carboxylate (1), and it was observed that 1 inhibits fibril growth with an IC₅₀ of 18 μM (FIGS. 2B and 2C). In contrast to EGCG, the fluorescence signal did not increase in the control reactions of 1 with no added fibril seeds (monomer only), indicating the absence of oligomer formation or spontaneous nucleation of fibrils over the 3-hour incubation period.

### Screening Additional Analogs Identifies Structure-Activity Relationships for Fibril Growth Inhibitors.

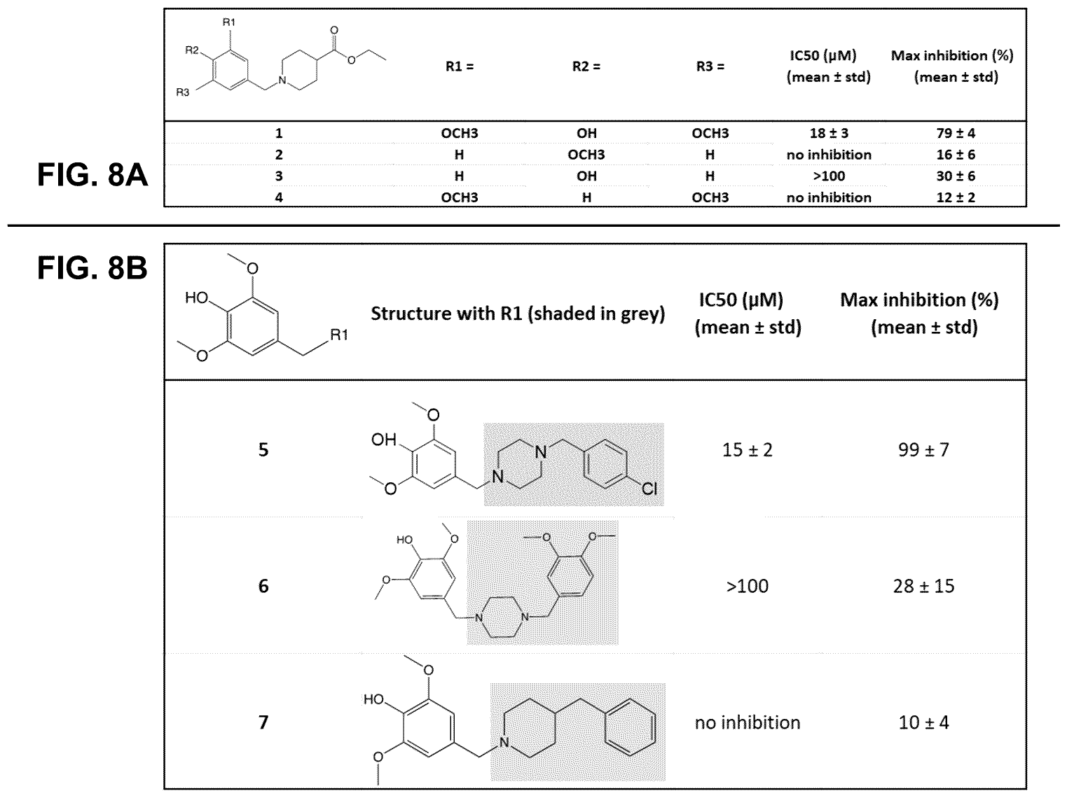

To determine whether the inhibition potency of compound 1 could be further improved, 29 analogs of 1 were examined in three rounds of structure-activity relationship (SAR) analysis. First, several analogs (2-4) with alternate substitutions in the benzyl group on the left-hand side of 1 (FIG. 8A, SAR1) were examined. A loss of inhibition potency was observed for all of these analogs. Next, when the piperidine in 1 was replaced with a piperazine and the ester was replaced with a chlorobenzyl-piperazinyl (5), the inhibition improved to 15 μM, and max inhibition was 99% compared to 18 μM and 79%, respectively, for 1. Other substitutions of 1 with another piperazinyl or piperidine (6 and 7) did not improve fibril growth inhibition (right side, SAR2, FIG. 8B). 23 piperazine analogs (8-30) with modification on the right-hand side were subsequently screened and several dimethoxyphenol piperazine compounds (8-13) that were more potent (lower IC50) and had greater maximum inhibition than 1 were found (Table 1).

TABLE 1
Compounds with highest inhibition potency after 3 rounds of structure-
activity screening.

| 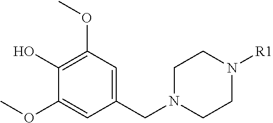 | Structure with R1 shaded | IC50 [95% Cl] [μM] [mean ± std] | Max Inhibition [%] [mean ± std] |
|---|---|---|---|
| 8 | 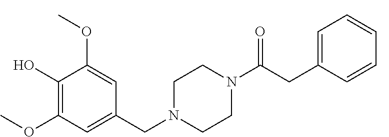 [SMILES: COc1cc(CN2CCN(C(=O)Cc3ccccc3)CC2)cc(OC)c1O] | 4 ± 1 | 86 ± 12 |
| 9 | 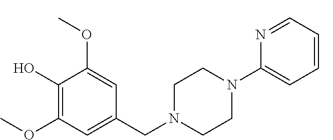 [SMILES: COc1cc(CN2CCN(c3ccccn3)CC2)cc(OC)c1O] | 7 ± 1 | 79 ± 8 |
| 10 | 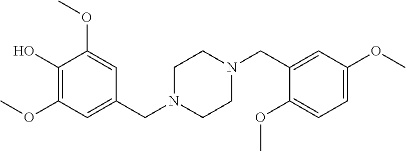 | 9 ± 3 | 90 ± 6 |
| 11 | 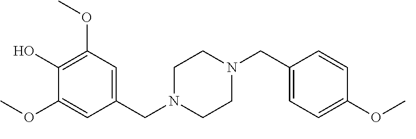 | 9 ± 4 | 76 ± 9 |
| 12 | 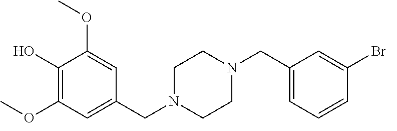 [SMILES: COc1cc(CN2CCN(Cc3cccc(Br)c3)CC2)cc(OC)c1O] | 12 ± 4 | 95 ± 3 |
| 13 | 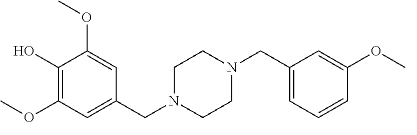 | 14 ± 2 | 84 ± 10 |
| 5 | 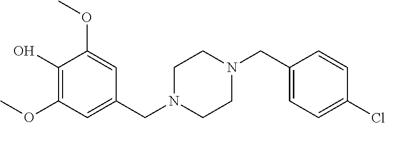 [SMILES: COc1cc(CN2CCN(Cc3ccc(Cl)cc3)CC2)cc(OC)c1O] | 15 ± 2 | 99 ± 7 |

The structures share a piperazinyl and a 4-hydroxy-3,5-dimethoxybenzylgroups. IC₅₀ is the mean±standard deviation (std) and maximum inhibition is calculated as a (Intensity_{100μM}-Intensity_{0μM})/intensity_{0μM} from three independent experiments.

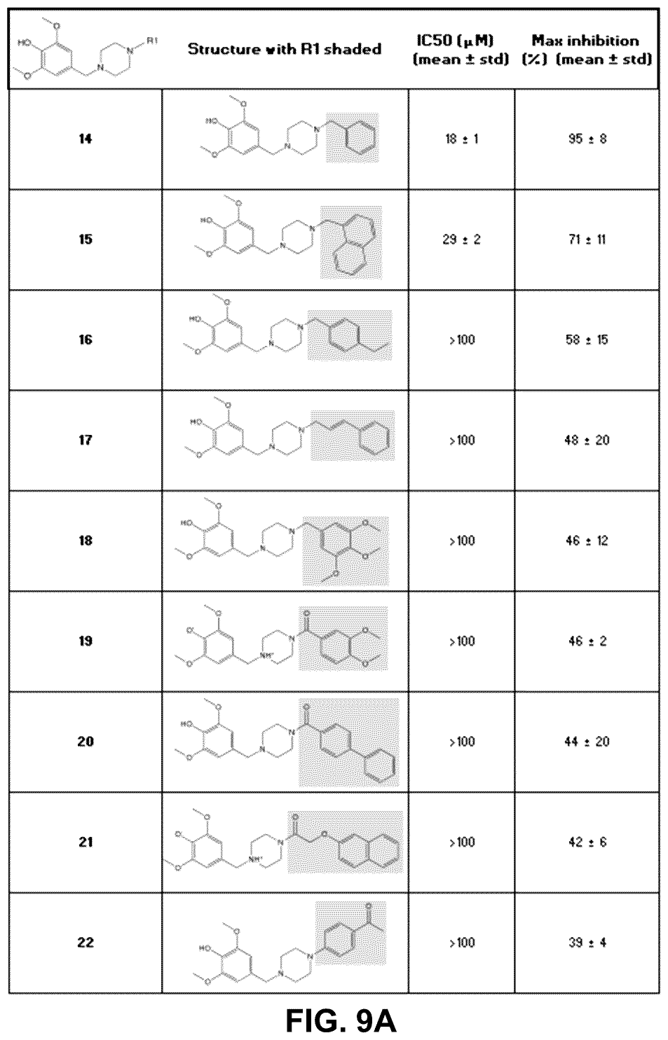

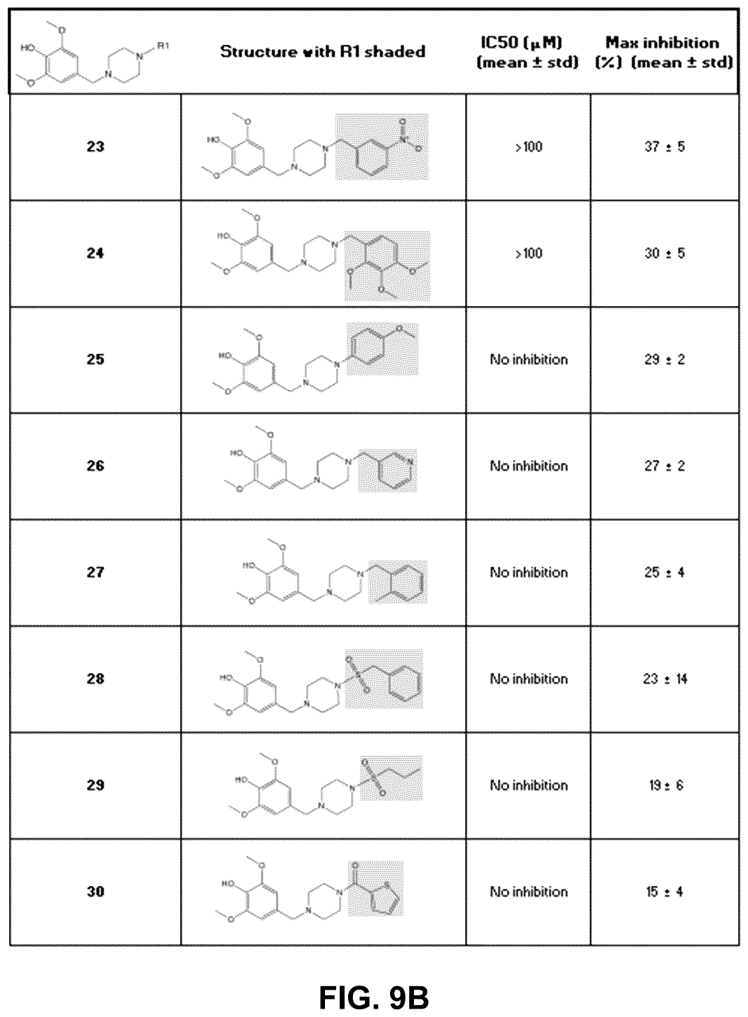

For example, 10, a piperazinyl dimethoxylphenol ethanedioate, had an IC₅₀ of 9±3 μM (FIGS. 2D and 2E). The analogs of 1 can be further divided into classes of phenacyl (8), pyridinyl (9), dimethoxy benzyl (10, FIG. 2D), methoxy benzyls (11 and 13), and halogen benzyls (5 and 12). Substitutions that included bulkier subgroups (14, 15, 18, 19, 20, and 21) and thiol-containing analogs (28, 29, and 30) had little inhibitory effect (FIG. 9(A-B)). It was also confirmed that 100 μM solutions of the test compounds had less than 15% higher native fluorescence relative to buffer alone, at the FlAsH excitation/emission wavelengths (Table 2).

TABLE 2
Native fluorescence measurements for the compounds.

| | % Fluorescence = 100% × [(Intensity_100 uM)/ |
|---|---|
| Compound | (Intensity_DMSObuffer)] (mean +/− std) |
| 2 | 89.0% +/− 0.2% |
| 3 | 90.0% +/− 1.6% |
| 4 | 97.5% +/− 2.1% |
| 5 | 99.2% +/− 1.4% |
| 6 | 97.6% +/− 1.7% |
| 7 | 99.4% +/− 1.1% |
| 8 | 91.1% +/− 0.0% |
| 9 | 94.2% +/− 0.4% |
| 10 | 102.7% +/− 0.2% |
| 11 | 100.3% +/− 0.4% |
| 12 | 99.4% +/− 0.9% |
| 13 | 95.8% +/− 0.5% |
| 14 | 99.1% +/− 0.3% |
| 15 | 108.1% +/− 0.3% |
| 16 | 106.5% +/− 1.0% |
| 17 | 94.1% +/− 1.7% |
| 18 | 100.5% +/− 0.7% |
| 19 | 102.2% +/− 0.5% |
| 20 | 91.5% +/− 1.4% |
| 21 | 97.0% +/− 1.5% |
| 22 | 98.5% +/− 0.0% |
| 23 | 91.5% +/− 0.9% |
| 24 | 100.4% +/− 0.7% |
| 25 | 101.7% +/− 1.3% |
| 26 | 92.9% +/− 0.7% |
| 27 | 103.6% +/− 2.6% |
| 28 | 94.2% +/− 0.1% |
| 29 | 97.4% +/− 0.1% |
| 30 | 110.7% +/− 6.0% |

Calculated percent native fluorescence for compounds (relative to buffer) in the absence of preformed fibrils. Percent native fluorescence is calculated as 100%×Intensity_{100μM}/intensity__{DMSObuffer}.

Whether apparent inhibitory effects were due to the fluorescence quenching of the FlAsH dye by the test compounds was further evaluated (Table 3). Only two of the compounds (6 and 15) had quenching of more than 25% fluorescence in the presence of fibrils pre-assembled in vitro with C2-aSyn monomers and incubated with FlAsH, but these two compounds showed minimal inhibition of fibril growth (FIGS. 8B and 9). This further confirmed that the inhibitory effects observed for the compounds were not due to quenching of FlAsH dye by the compounds at higher concentrations.

TABLE 3
Fluorescence quenching measurements for the compounds.

| | % Quenching = 100% − {[(Intensity_100 uM)/ |
|---|---|
| Compound | (Intensity_0 uM)] × 100%} (mean +/− std) |
| 2 | 9.2% +/− 9.9% |
| 3 | 12.3% +/− 0.9% |
| 4 | 9.8% +/− 3.2% |
| 5 | 8.7% +/− 3.0% |
| 6 | 25.8% +/− 0.5% |
| 7 | 8.3% +/− 1.8% |
| 8 | 7.0% +/− 7.8% |
| 9 | 3.6% +/− 7.3% |
| 10 | −3.2% +/− 1.9% |
| 11 | 0.8% +/− 2.7% |
| 12 | 11.3% +/− 7.6% |
| 13 | 14.6% +/− 3.5% |
| 14 | 5.1% +/− 5.6% |
| 15 | 28.1% +/− 0.8% |
| 16 | 9.2% +/− 2.0% |
| 17 | 5.4% +/− 0.9% |
| 18 | 8.7% +/− 6.0% |
| 19 | 10.3% +/− 2.7% |
| 20 | 11.1% +/− 9.5% |
| 21 | 6.7% +/− 5.1% |
| 22 | 1.5% +/− 3.1% |
| 23 | 24.2% +/− 7.2% |
| 24 | 2.3% +/− 1.5% |
| 25 | 10.0% +/− 2.6% |
| 26 | 7.2% +/− 1.7% |
| 27 | 6.2% +/− 1.5% |
| 28 | 17.2% +/− 2.9% |
| 29 | 8.6% +/− 6.6% |
| 30 | 1.4% +/− 2.9% |

Calculated percent quenching for compounds in the presence of preformed C2-aSyn fibrils and FlAsH dye. Percent quenching is calculated as 100%−(Intensity100 μM/Intensity0 μM)×100%.

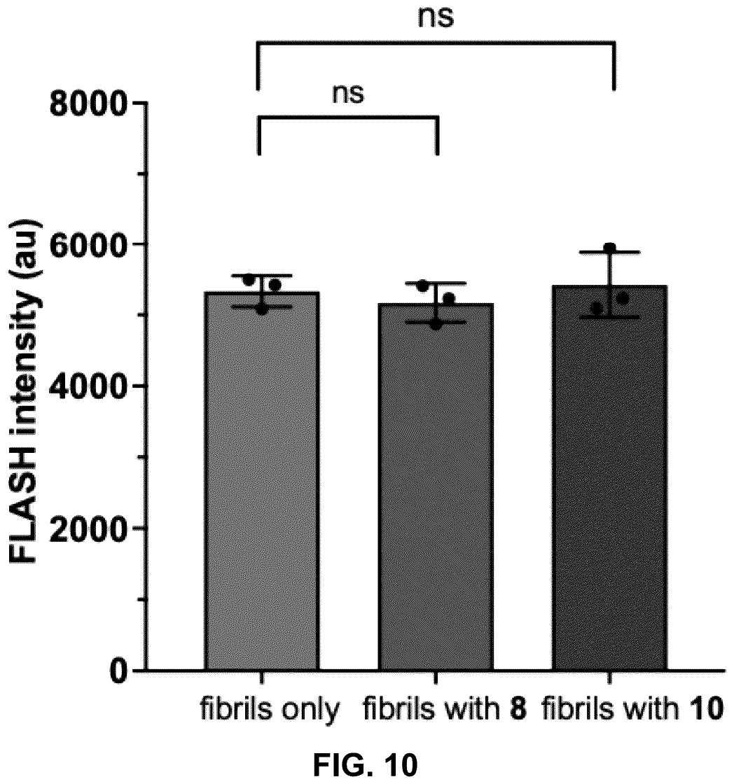

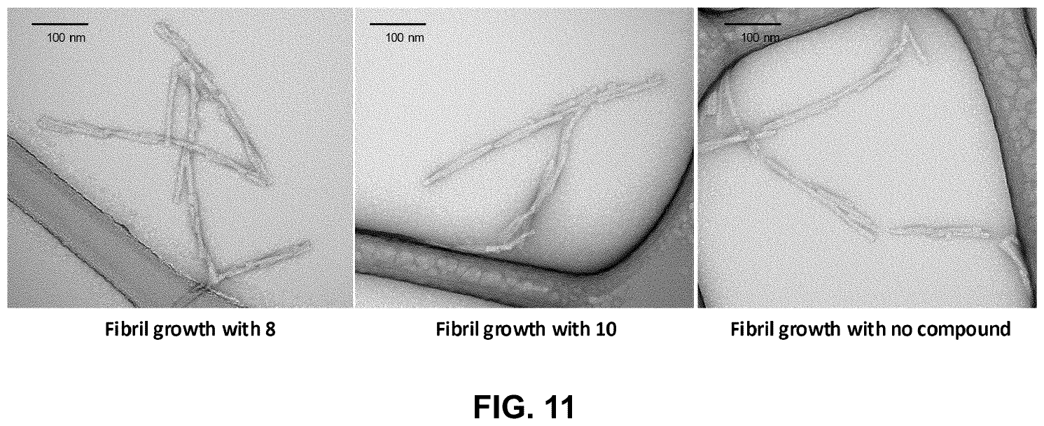

No change in FlAsH fluorescence was observed after C2-aSyn fibrils were incubated with the compounds in the absence of monomer for 3 hours, suggesting that these compounds do not disrupt or disaggregate the fibrils (FIG. 10). Fibril growth products of the lead compounds 8 and 10 after growth were further examined with negative stain transmission electron microscopy (TEM). Similar aSyn fibril morphology was observed in the absence and presence of the two lead compounds (FIG. 11). Given the variable aSyn fibril density captured on grid, it was unable to quantify fibril concentration differences.

### Fibril Growth Inhibition Confirmed by Conformation-Specific Immunoassay

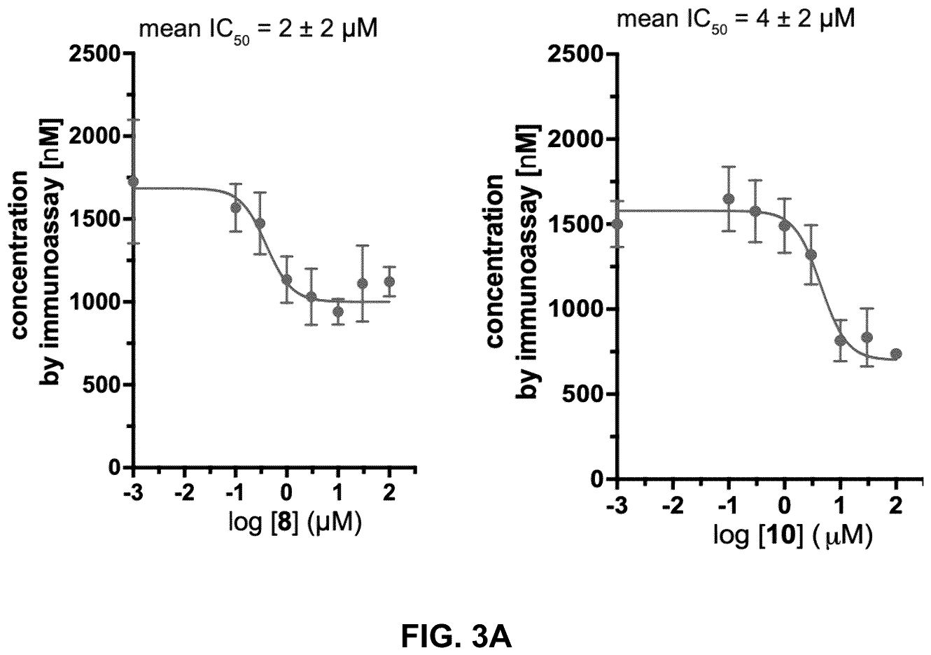

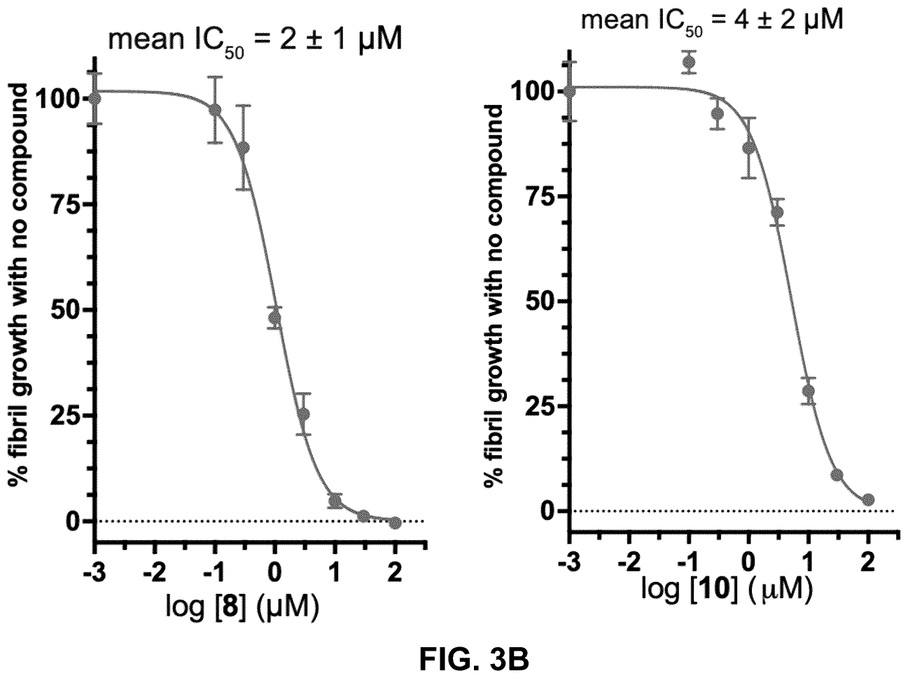

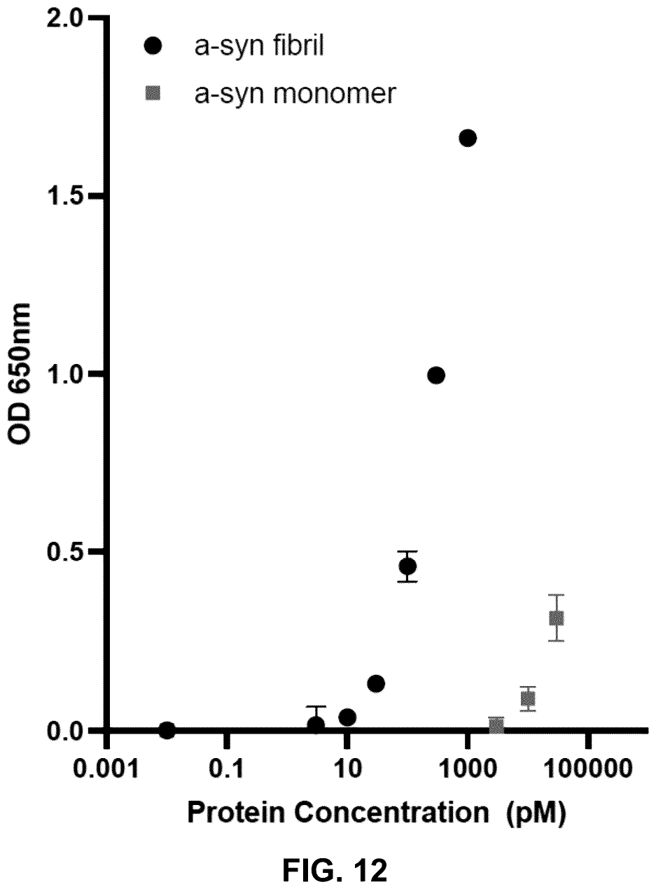

To confirm the inhibition of fibril growth for compounds 8 and 10, an orthogonal fibril growth assay that does not rely on fluorescence was used. A sandwich enzyme-linked immunosorbent assay (ELISA) was developed that utilizes a conformation-specific antibody (Abcam 209538, MJFR-14-6-4-2) in the capture step and an in-house monoclonal antibody 13G5 to measure fibril concentration at the end of the growth assays. This immunoassay is approximately 300-fold more sensitive for detecting fibrils than monomers (FIG. 12). In contrast to the FlAsH assay, this immunoassay measures the level of fibril seeds at baseline, and growth is measured as an increase in signal relative to the baseline signal of fibril seeds. The incubation time for the fibril growth reaction was increased to 20 hours and the aSyn monomer concentration was increased to 6 μM, in order to increase the amount of fibril growth and compensate for the reduced sensitivity of the immunoassay relative to the FlAsH assay. Under these conditions, fibril concentrations increase by 70% to 100% over 20 hours in the absence of inhibitor compounds. It was confirmed that the presence of the compound decreased fibril growth as measured by the ELISA in a concentration-dependent manner for compounds 8 and 10 (FIG. 3A). FlAsH fluorescence measurements in parallel reactions showed similar inhibition patterns (FIG. 3B). It is possible that during these extended incubation times, the ELISA may be measuring a combination of molecular events that increase fibril concentration, including elongation as well as primary or secondary nucleation. However, in this context, the orthogonal assay provides confirmation that compounds 8 and 10 inhibit fibril accumulation and that the FlAsH assay can successfully identify inhibitory compounds.

### Fibril Growth Inhibition Observed by ThioT Fluorescence

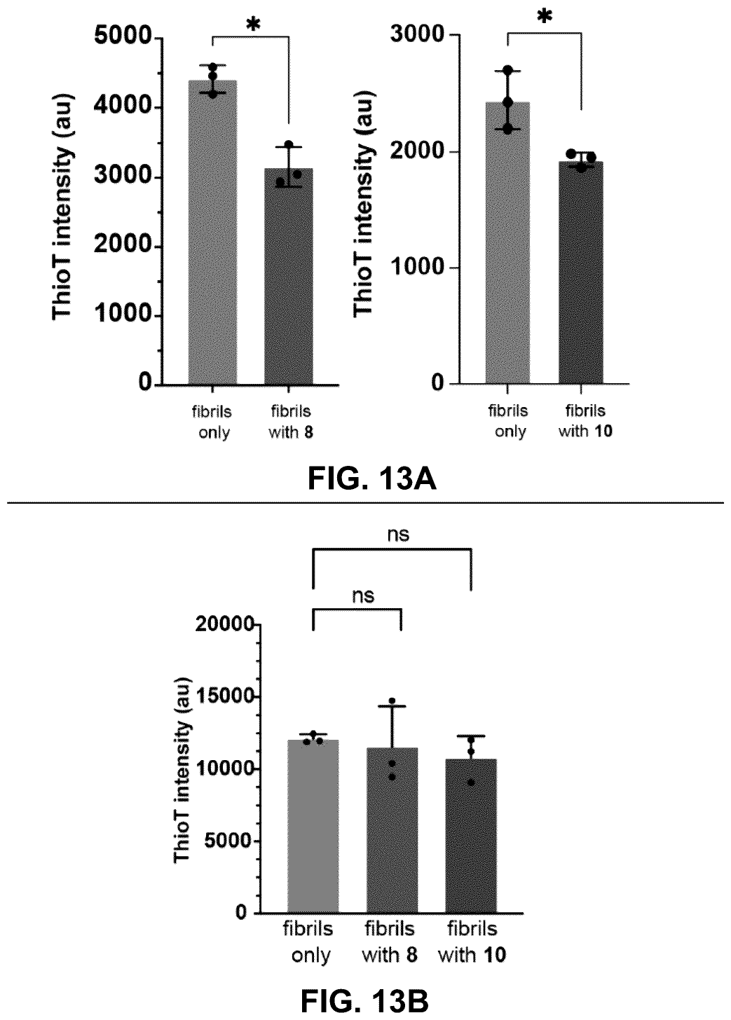

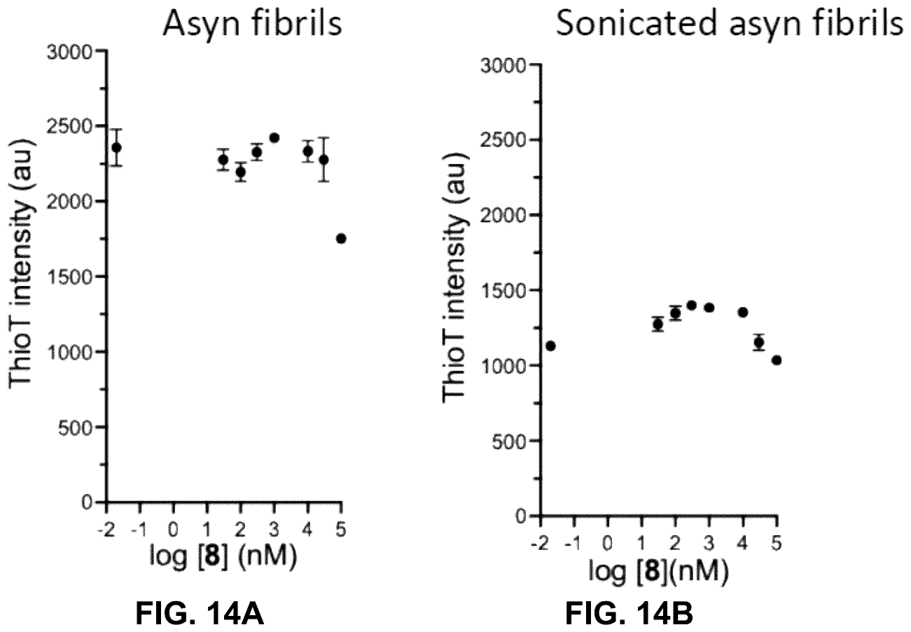

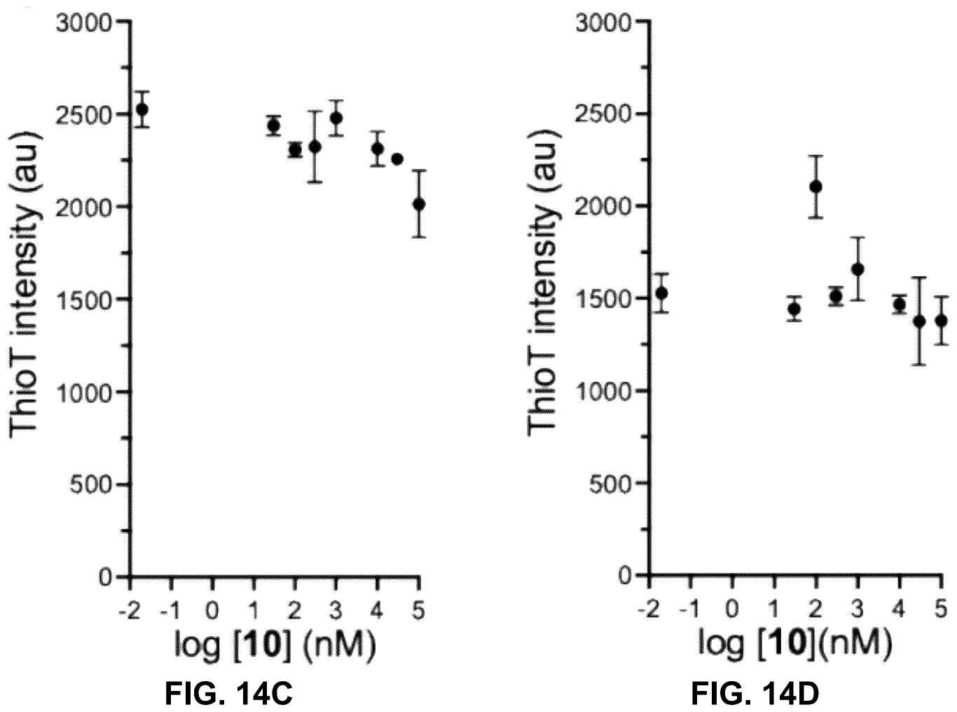

Whether fibril growth inhibition by the lead compounds can be measured with ThioT fluorescence, which has been widely used in previous studies of fibril growth inhibitors, was further evaluated. Due to the low sensitivity of ThioT, the fibril seed concentration was increased to 3 μM and continuous shaking was utilized for 20 hours to achieve measurable fibril growth. In the initial control experiments, it was observed that compounds 8 and 10 reduced ThioT fluorescence in the presence of fibrils (FIG. 13A), potentially due to either quenching or competition with ThioT binding to fibrils. In competition binding assays measuring ThioT fluorescence in the presence of fibrils (FIG. 14), a small but significant decrease in ThioT fluorescence was observed at high concentrations of fibrils, but it was not able to be determined whether this is related to competitive displacement of ThioT or alternatively fluorescence quenching mediated by compounds interacting with fibrils at distinct binding sites. It was also not observed that increasing the concentration of fibril ends by sonicating the fibrils altered the effect of the compounds on ThioT fluorescence, indicating that it is not mediated by competition between ThioT and compounds at the ends of the fibrils.

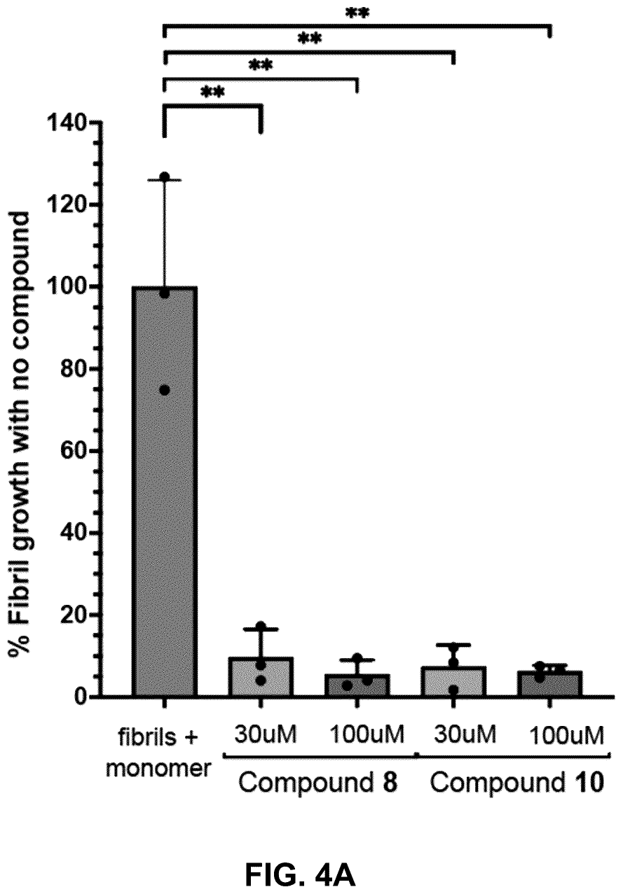

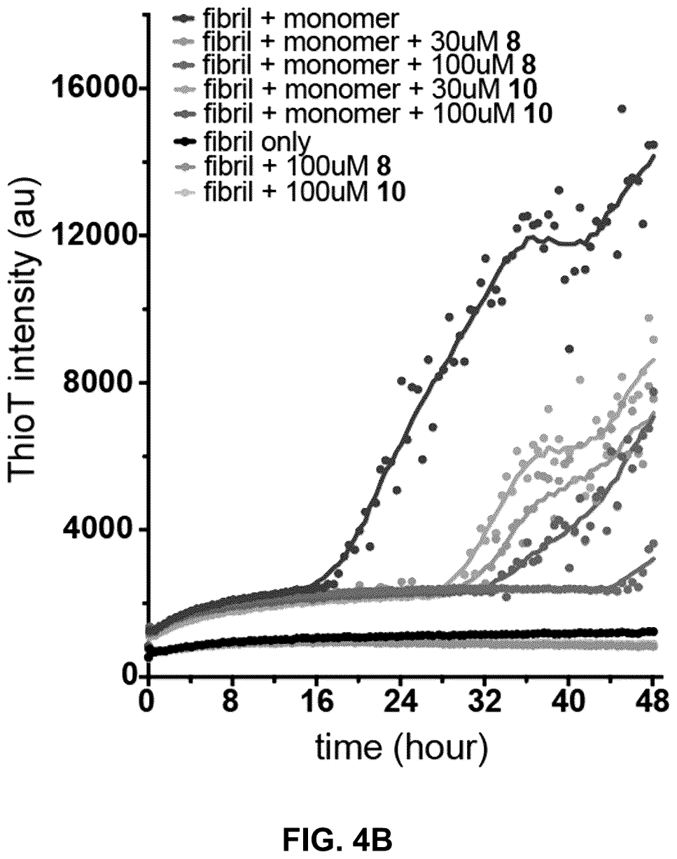

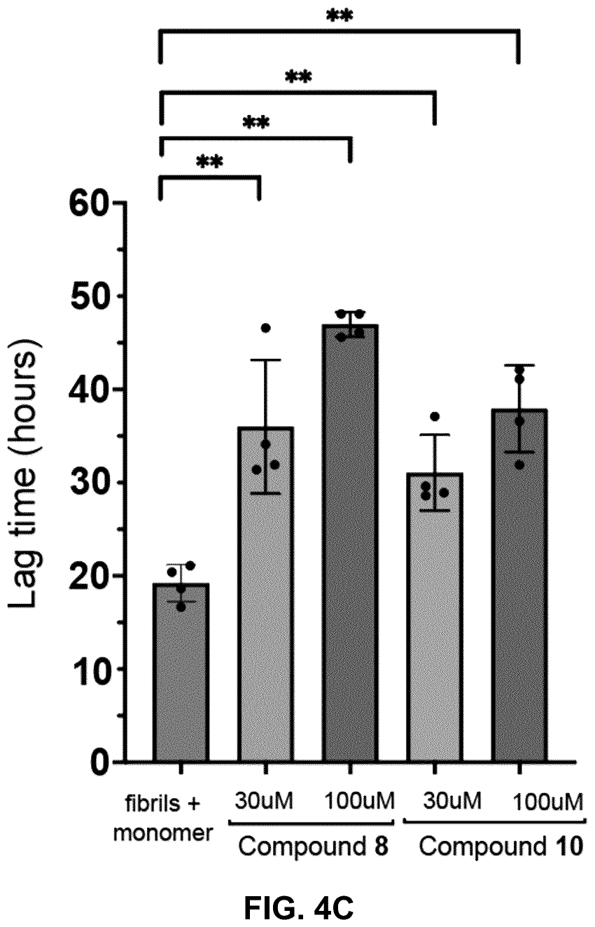

To address the potential for this effect to interfere with measurements of fibril growth, centrifugation and washing was used to isolate fibrils from compounds following the growth reaction. The fibril pellet was then resuspended in buffer containing ThioT (18 μM) to measure fibril growth. This protocol substantially reduced the quenching or inhibitory effect of the compounds on ThioT fluorescence (FIG. 13B). It was observed that compounds 8 and 10 significantly inhibited fibril growth (FIG. 4A).

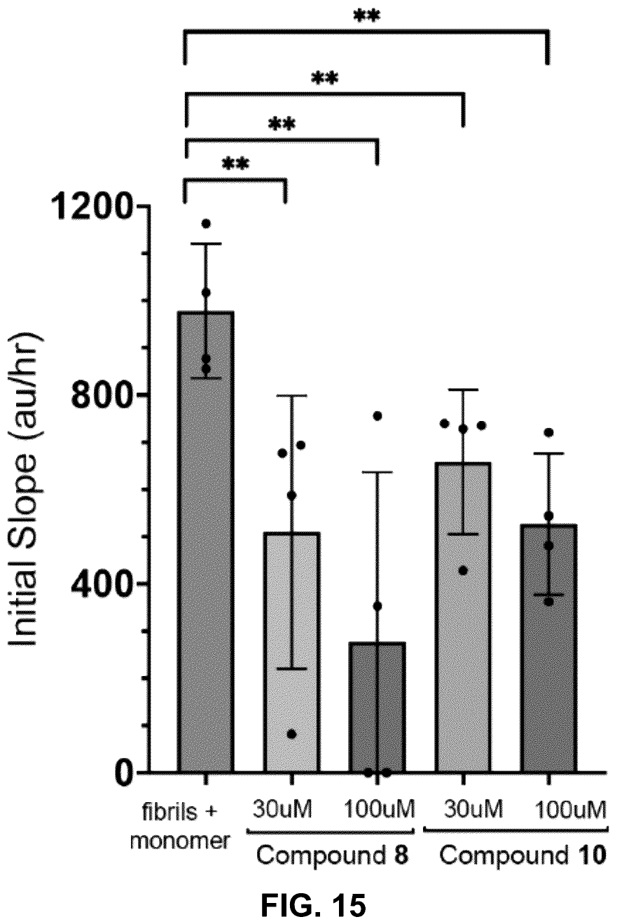

Given the reliance of this assay on endpoint ThioT measurements, a kinetic fibril growth assay was next performed with 1 μM fibril seeds, 6 μM aSyn monomer and ThioT, utilizing continuous shaking in a plate reader in the presence or absence of compound. Under these assay conditions, linear fibril growth based on ThioT fluorescence was not observed, but instead a lag phase followed by a substantial rise in ThioT fluorescence was observed, suggesting a rapid increase in fibril growth, followed by a plateau. It was observed that compounds 8 and 10 inhibit both the duration of the lag phase as well as the rate at which ThioT fluorescence increases following the longer lag period (FIGS. 4B, 4C, and 15).

Dimethoxyphenyl Piperazine Compounds Also Inhibit the Growth of Fibrils Amplified from LBD Postmortem Brain Tissue

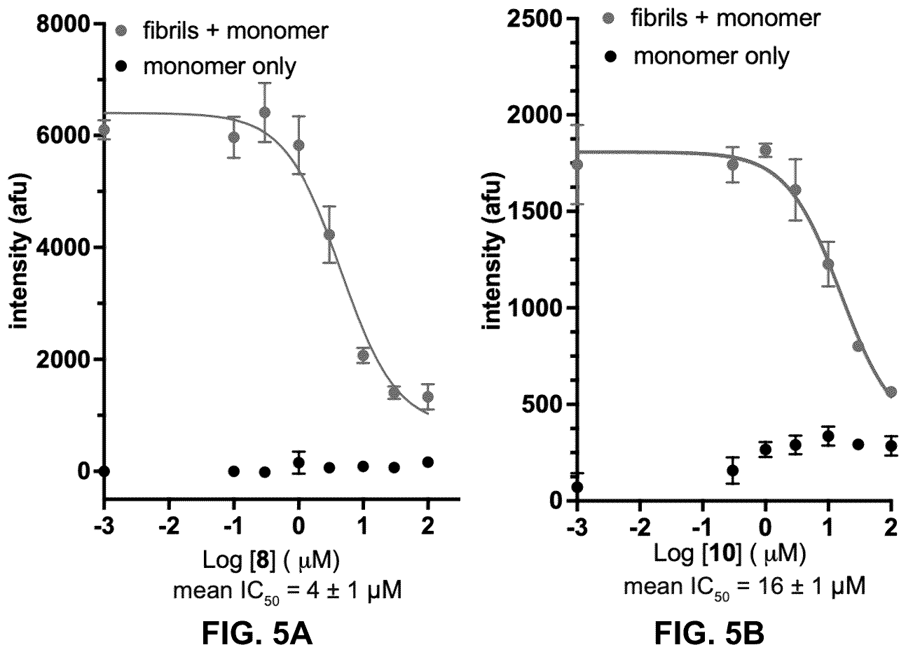

Emerging studies have shown that the structure of aSyn fibrils derived from postmortem LBD brain tissue differs substantially from the structures of fibrils produced in vitro from recombinant protein. Differences in structure among these distinct fibril conformers may affect the binding and inhibition of fibril growth by small molecule compounds. Compounds 8 and 10 were initially identified based on in vitro fibrils. Amplified LBD fibrils were recently generated using fibrils extracted from LBD brain tissue, and it was shown that the fold of the LBD amplified fibrils matched the fold of aSyn fibrils directly extracted from PD and LBD brain tissue. Amplified LBD fibril seeds were used in the same fibril growth assay to test these lead analogs. Compounds 8 and 10 showed similar IC₅₀ values to those obtained for in-vitro assembled fibril seeds (FIG. 5). This result indicates that compounds 8 and 10 can inhibit the growth of fibrils that are structurally similar to those accumulating in PD and LBD.

### Docking Studies Suggest a Potential Mechanism for Fibril Growth Inhibition.

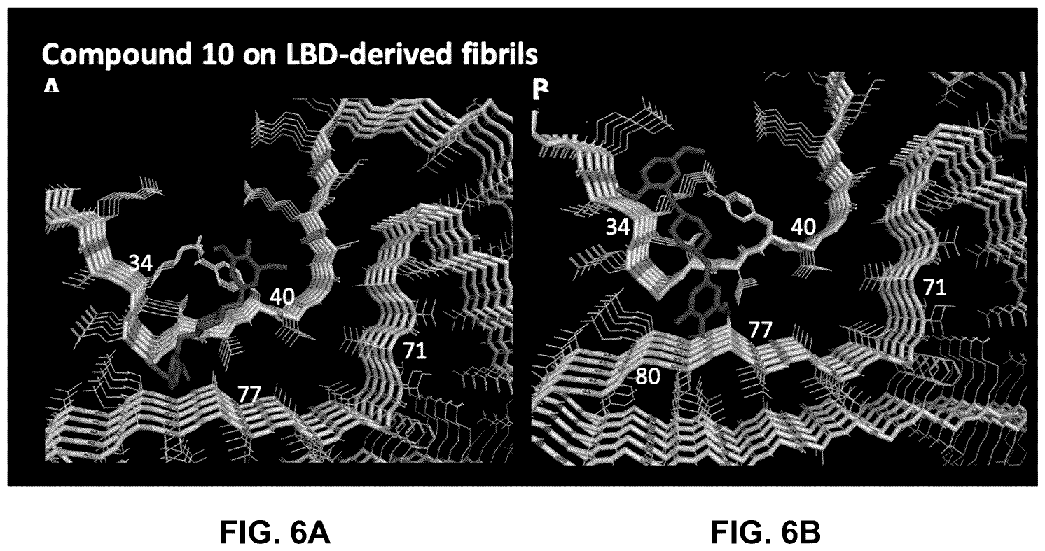

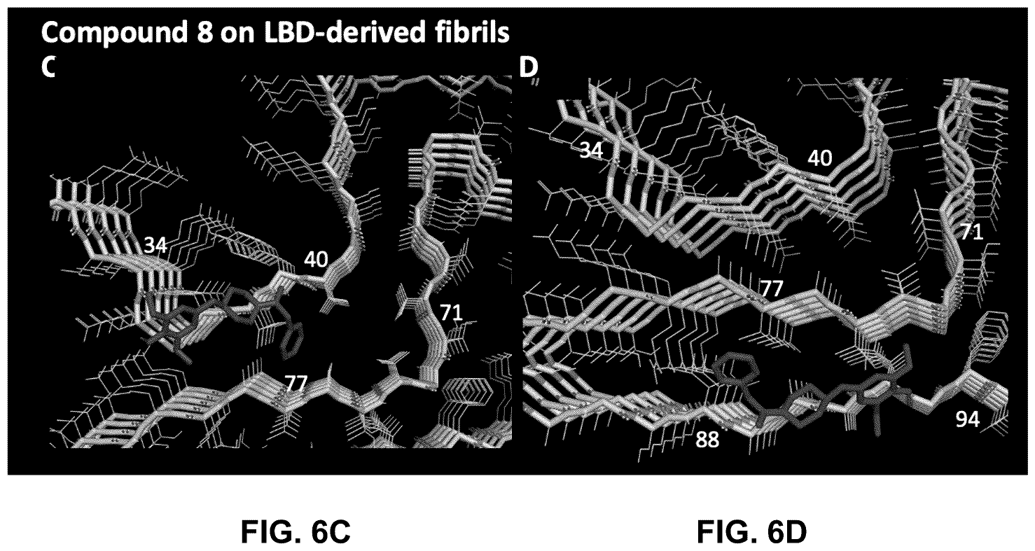

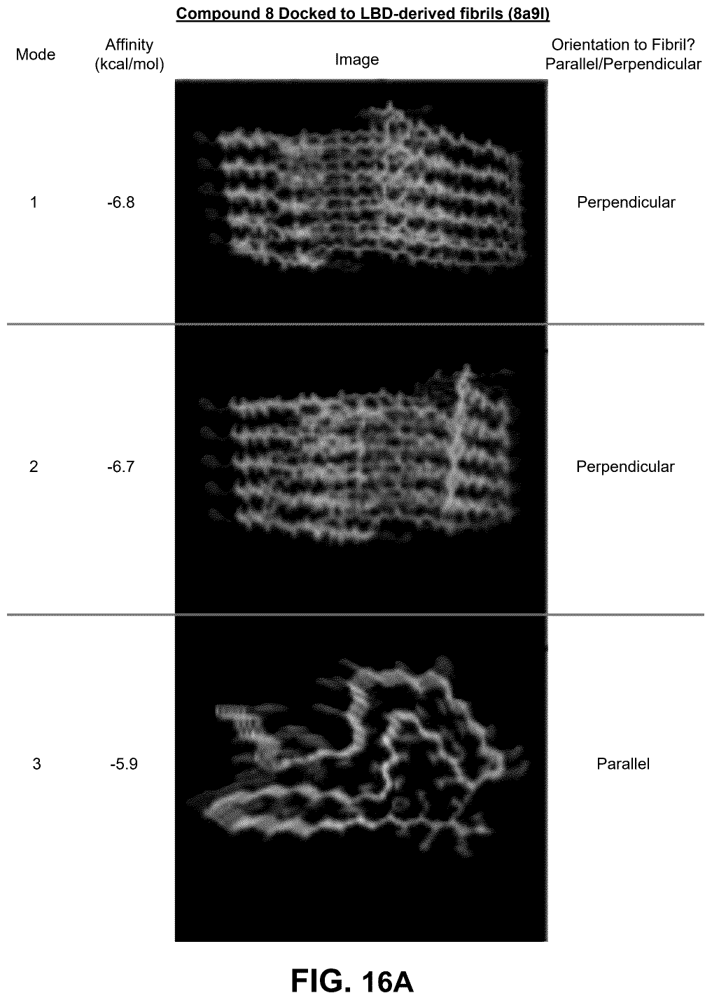

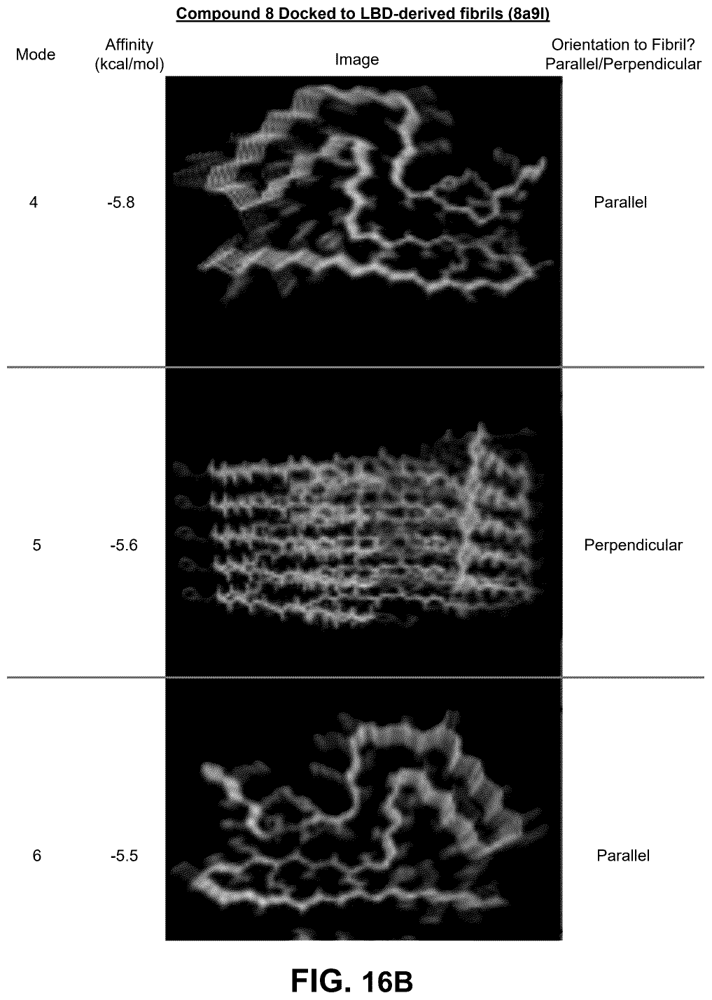

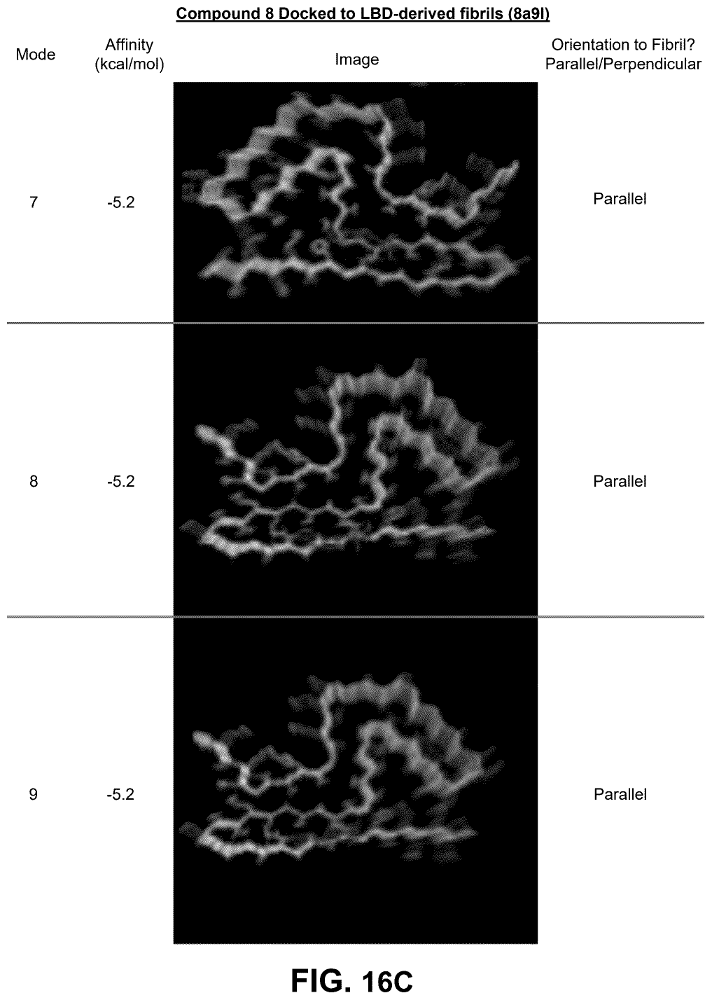

Small molecule compounds can potentially bind to fibrils or monomeric proteins in many different ways. When binding to the fibrils, the small molecule can bind to the highly ordered beta sheet region or, less likely, to the disordered N-terminal or C-terminal regions. To explore potential modes of interaction for inhibitor compounds, molecular docking software, Autodock Vina, was utilized to predict potential binding sites of the inhibitor compounds on LBD aSyn fibrils (FIGS. 6 and 16). LBD fibrils (PDB ID: 8a9I) were focused on as a docking substrate since this structure was determined for aSyn fibrils extracted from postmortem human brain tissue. SSNMR studies indicated that the structure of amplified LBD fibrils (PDB ID: 8fpt) is highly similar to 8a9I, but were not able to resolve the structural conformation of the E46-V66 region. To maximize the effect of observing binding to fibril ends in parallel or perpendicular to the fibril axis, the docking substrate was limited to 5 fibril units, which is sufficient to also identify binding sites spanning multiple monomeric units along the fibril surface. According to some embodiments of the present disclosure, a structural model for in vitro aSyn fibrils is refined based on single particle cryo-EM and solid-state NMR data.

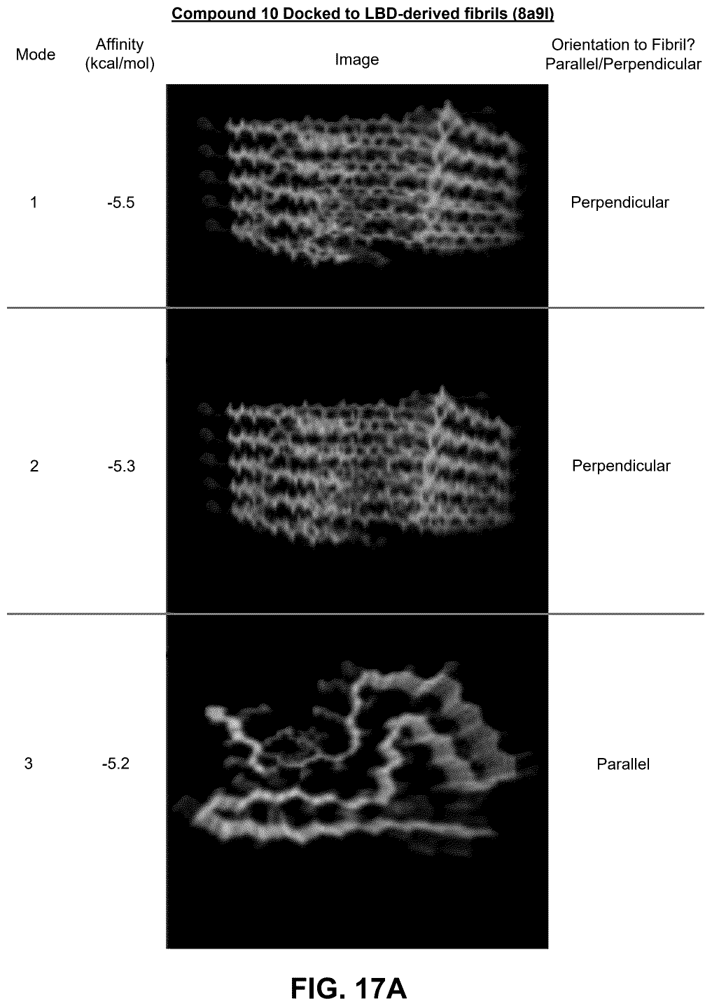

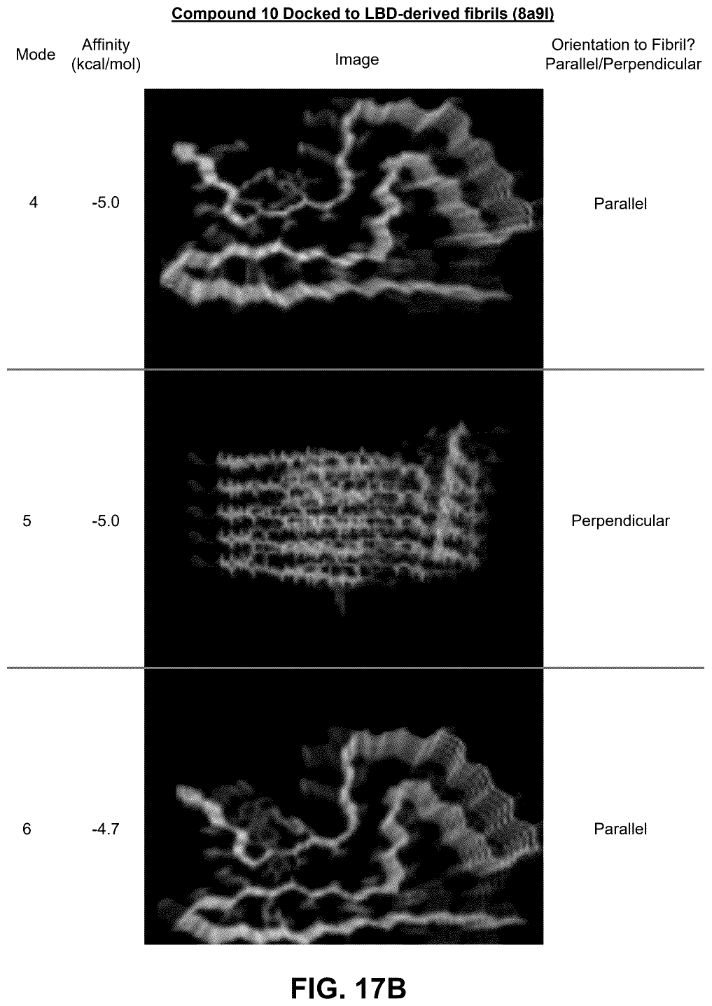

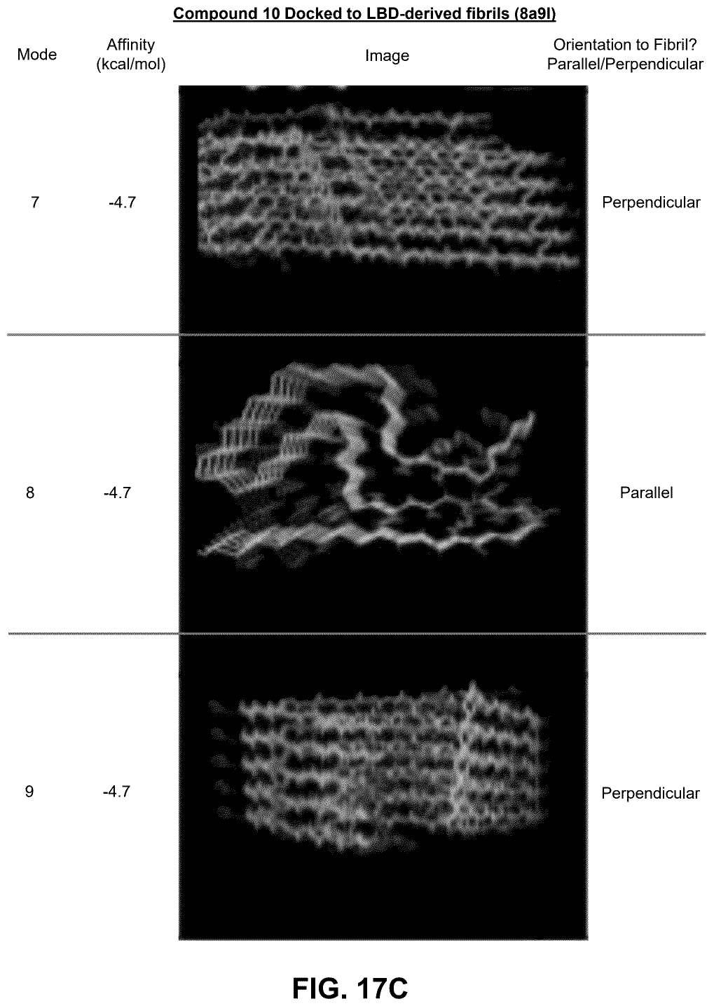

Among binding sites with the lowest predicted binding energy for the two example compounds, several poses were observed on the surface of the elongating ends of the fibrils, which indicate a “capping” mechanism of inhibition where the compounds compete with the binding of monomeric aSyn during elongation. Six of the nine docked sites for 8 and four of the nine sites for 10 are located on the surface of the elongating end of the fibril (FIG. 14). Interestingly for these binding sites, the compounds interact with one beta-strand and are oriented along the peptide backbone. The piperazine interacts with the backbone, while the aromatic groups on the left-hand and right-hand sides interact with the backbone and side chains. The interaction of the compounds with the elongating end may constrain the ability of additional monomers to align with the beta sheet conformation of the fibril seed, thus decreasing the rate of monomer association required for fibril growth. The alternative docked binding sites are along the fibril surface, parallel to the z axis, spanning 3 to 4 monomeric units (FIG. 16(A-C) and FIG. 17(A-C)). Moreover, in some of these binding sites that are parallel to the z axis, the methoxy subgroups of the aromatic groups (left-hand or right-hand side) interact with fibril ends, which may also constrain the ability of additional monomers to attach to the elongating end.

### Methods and Materials

### Compounds

Compounds were purchased from Chembridge (San Diego, California USA) and Vitas-M Laboratory (Hong Kong). They were dissolved in DMSO at a concentration of 10 mM and stored at −20° C.

Preparation of Recombinant WT-aSyn, C2-WT-aSyn and aSyn Fibrils

Untagged WT-aSyn monomer, C2-tagged WT-aSyn monomer, and aSyn fibrils were prepared as described. Briefly, aSyn protein was expressed in E. Coli (BL21-CodonPlus (DE3)-RIL Competent Cells, Agilent Catalog: 230245) using the pRK172 plasmid expressing the aSyn sequence. Bacteria were grown in TB broth containing 50 μg/ml ampicillin overnight with shaking. Asyn monomeric protein was extracted using osmotic shock followed by heat denaturation and ion-exchange chromatography. Purified aSyn monomer was stored at -80° C. until use.

WT-aSyn and C2-WT-aSyn fibrils were made by incubating 2 mg/ml of WT-aSyn protein or C2-WT-aSyn protein, respectively, in 20 mM Tris-HCl, pH 8.0 plus 100 mM NaCl buffer with shaking at 1000 rpm in an Eppendorf thermomixer set to 37° C. Fibrils were stored at 4° C. until use. The concentration of aSyn monomer and fibrils was determined using a micro-BCA assay performed according to the manufacturer's instructions. Briefly, the micro-BCA assay (Thermo Fisher, Catalog number 23235) was utilized to estimate the amount of fibrils. A standard curve (200 μg/ml to 0.5 μg/ml) using manufacturer supplied diluted albumin (BSA) standard was used to determine concentration of aSyn. The working reagent was made by mixing 25 parts of Micro BCA Reagent MA, 24 parts Reagent MB, and 1 part of Reagent MC (25:24:1, Reagent MA:MB:MC). In a microplate well, 150 μL of each standard or unknown sample (three replicates) was added along with 150 μl of the working reagent. The plate was incubated for 2 hours at 37° C., cooled for 10 minutes and absorbance measured at 562 nm on a plate reader. The amplified fibrils were centrifuged at 21,000 g for 15 min at 4° C. to separate fibrils from monomer. The supernatant and total sample containing fibrils plus unincorporated monomer was also assessed. The measured decrease in aSyn monomer concentration between total and supernatant was used to determine the concentration of fibrils.

### Preparation of LBD Amplified Fibrils

The Movement Disorders Brain Bank, Washington University, St. Louis, MO, provided well-characterized postmortem frozen brain tissue of de-identified participants. Written informed consent to perform a brain autopsy for research purposes was obtained from participants of the Movement Disorders Brain Bank. After death, the immediate next-of-kin were contacted and confirmed consent for brain removal and retention of brain tissue for research purposes. The participant who donated brain tissue used in these experiments participated in a clinical study approved by the Human Research Protection Office at Washington University prior to autopsy. All methods using human autopsy-derived material were carried out in accordance with relevant guidelines and regulations.

Fibril growth inhibition assays used amplified fibrils from case LBD1. Human postmortem brain tissue samples were sequentially extracted in buffers of increasing detergent strength using a Dounce Homogenizer to generate LBD-derived insoluble fraction seeds. The LBD seeds were combined with purified aSyn monomer and subjected to 6 cycles of fragmentation followed by quiescent incubations to generate amplified fibrils.

### Fibril Growth Assay Utilizing FlAsH Fluorescence Measurements

The fibril growth assay was performed similarly to previously published methods. Briefly, fibril samples at 1 μM concentration in fibril buffer were either sonicated for 5 min at amplitude 50 in a cup horn sonicator (Qsonica 600) for the recombinant WT- or C2-fibrils for 1 minute at amplitude 50 for the LBD-derived fibrils and mixed with 3 μM of C2-aSyn monomer and compound at a range of concentrations in 20 mM Tris-HCl pH 8.0, 100 mM NaCl buffer with 50 μL total volume. Compounds were dissolved in DMSO and the final concentration of DMSO was kept constant at 1.25%. The seeds, aSyn monomer, and various concentrations of the compounds of interest were quiescently incubated for 3 hours at 37° C. in Corning Black 96-well plates (Fisher, catalog no. 07-200-762). After 3 hours, a 50 μL FlAsH assay mixture consisting of 7 mM tris(2-carboxyethyl)phosphine, 2 mM EDT, 2 mM EDTA, 50 nM FlAsH-EDT2 (Invitrogen TC-FlAsH II in-cell tetracysteine tag detection kit, catalog no. T34561), and 400 mM Tris-HCl, pH 8.0 was added and the total 100 μL mixture incubated for additional 1 hour at room temperature. FlAsH fluorescence was detected in a BioTek plate reader using a 485/20-nm excitation filter, a 528/20-nm emission filter, top 510-nm optical setting, and gain setting 100. Measurements were performed in triplicates. Dose-dependent curve fit a four-parameter logistic curve, Y=Bottom+(Top−Bottom)/(1+10\hat{ }((Log IC₅₀−X)*HillSlope)) to determine IC₅₀. Mean IC₅₀ was calculated from 2 to 3 experiments performed on different days. Maximum inhibition is calculated as (Intensity_{100μM}−Intensity_{0μM})/Intensity_{0μM}×100% where Intensity_{100μM} and Intensity_{0μM} is the intensity at 100 μM of compound and intensity with no compound in the presence of fibrils and monomers.

### Measurement of Quenching

Experiments to evaluate quenching were performed with 1 μM fibrils assembled from C2-WT-aSyn plus 100 μM compound in the absence of monomer. Fibril seeds and compound were incubated for 30 minutes in 20 mM Tris-HCl pH 8.0, 100 mM NaCl buffer (50 μL total volume), and then 50 μL FlAsH fluorescence assay mixture was added as above and incubated for an additional 1 hour at room temperature. FlAsH fluorescence was detected in a BioTek plate reader using a 485/20-nm excitation filter, a 528/20-nm emission filter, top 510-nm optical setting, and gain setting 100. Percent quenching is calculated as 100%−(Intensity_{100 μM}/Intensity_{0μM}).

### Measurement of Native Fluorescence

Experiments to evaluate native fluorescence were performed with 100 μM compound alone in the absence of fibril seeds and monomer. In a similar manner, compounds were incubated for 30 minutes in 20 mm Tris-HCl pH 8.0, 100 mM NaCl buffer (50 μL total volume) and then 50 μL FlAsH fluorescence assay mixture was added as above and incubated for an additional 1 hour at room temperature. FlAsH fluorescence was detected in a BioTek plate reader using a 485/20-nm excitation filter, a 528/20-nm emission filter, top 510-nm optical setting, and gain setting 100. Percent native fluorescence is calculated as 100%×Intensity_{100μM}/intenSity_{_DMSObuffer}, with Intensity_{100μM} as the intensity of 100 μM compound in the absence of monomer and fibrils and intensity_{_DMSObuffer} is the background intensity of buffer.

### Sandwich ELISA

Sandwich ELISA was performed. Briefly, anti-alpha-synuclein antibody [MJFR-14-6-4-2](Abcam ab209538) was used as capture antibody and diluted to 1 μg/mL in 34 mM sodium bicarbonate, 16 mM sodium carbonate solution with 3 mM sodium azide. The capture antibody solution was added to a 96-well plate (50 μL/well) and incubated overnight at 4° C. Plates were washed 5× with 150 μL 1XPBS+0.05% Tween-20/well in between each step. Plates were blocked with 150 μL of 2% BSA (Sigma-Aldrich #A7906) in PBS at 37° C. for 2 hours. Before standard and sample addition, plates were washed as previously stated. Samples were diluted at 1:10,000 in sample buffer containing no SDS to reach an SDS concentration of 0.06%. Standards and samples were added to the plate 50 μL/well in duplicate wells and incubated overnight at 4° C. with slow rotation. The detection antibody (13G5B, in-house) solution was diluted to a final concentration of 1 μg/mL in PBS+0.05% BSA+0.05% Tween. The detection antibody solution was added 50 μL/well and incubated at 37° C. for 2 hours. Streptavidin poly-HRP80 (Fitzgerald #65R-S118) was diluted 1:8000 in 1% BSA in 1XPBS+0.05% Tween-20. The streptavidin solution was added 50 μL/well and incubated for 90 minutes at room temperature in the dark. The plate was washed as described previously. Fifty μL/well of 3,3′,5,5′-Tetramethylbenzidine Liquid Substrate, Super Slow (Sigma #T5569) was rocked on the rotator until the first reading. Readings were done on Synergy 2 (BioTek) at 650 nM at 9, 12, and 15 minutes. The time point with the highest ratio between the top concentration and 0 concentration (background) in the standard curve was chosen for data analysis. Dose-dependent curve fit a four-parameter logistic curve, Y=Bottom+(Top−Bottom)/(1+10\hat{ }((Log|C₅₀−X)*HillSlope)) to determine IC50. Mean IC50 calculated from 2 experiments.

### ThioT Fluorescence Assay for Fibril Growth

The first ThioT fibril growth assay was performed similarly to that of the FlAsH fluorescence assay with a few modifications. Fibril samples (3 μM concentration) were sonicated for 5 min at amplitude 50 in a cup horn sonicator (Qsonica 700) and mixed with 6 μM of C2-aSyn monomer and compound at 30 μM and 100 μM in 20 mm Tris-HCl pH 8.0, 100 mm NaCl buffer with 100 μL total volume. Compounds were dissolved in DMSO and the final concentration of DMSO was kept constant at 1.25%. The seeds, aSyn monomer, and concentrations of the compounds of interest were incubated for 20 hours at 37° C. in Eppendorf Thermomixer R at 1000 rpm in ultracentrifuge tubes. After 20 hours, the assay mixture volume (100 μL) was increased to 300 μL with fibril buffer (20 mM Tris-HCl pH 8.0, 100 mM NaCl buffer) and centrifuged for 100,000×g for 20 min at 4° C. The supernatant (270 μL) was removed and replaced with 270 μL fibril buffer. 300 μL total volume was again centrifuged at 100,000×g for 20 min at 4° C. Again, the supernatant (270 μL) was removed, the seeds were recovered and mixed with 50 μL fibril buffer with 0.1% Triton. The 80 μL sample was placed into a Corning Black 96-well plates (Fisher, catalog no. 07-200-762) and incubated with 18 μM ThioT in Fibril Buffer. A total volume of 160 μl/well was incubated at room temperature for 24 hours at 200 rpm in a Benchmark Scientific Incumixer. After 24 hours, ThioT fluorescence was measured in a BioTek plate reader using a 440/30-nm excitation filter, a 485/20-nm emission filter, and top 50% optical setting. % Fibril Growth without compound is calculated by (Intensity Measurement−Intensity_fibrils_only)/(Intensity_nocompound−Intensity_fibril_only), where Instensity_fibril_only is the average intensity of fibrils in the absence of monomer and compounds, Intensity_nocompound is the average intensity of fibrils incubated with monomers in the absence of compounds.

The kinetic seeded ThioT fibril growth assay was performed similarly except that 1 μM in vitro aSyn fibrils were sonicated for 5 min at amplitude 50 in a cup horn sonicator (Qsonica 700) and mixed with 6 μM of C2-aSyn monomer, compound at 30 μM and 100 μM in 20 mm Tris-HCl pH 8.0, 100 mm NaCl buffer, and 18 μM ThioT with 100 μL total volume in a Corning Black 96-well plates (Fisher, catalog no. 07-200-762) and. Compounds were again dissolved in DMSO and the final concentration of DMSO was kept constant at 1.25%. The compound, monomer, aSyn fibril and ThioT mixtures were pre-incubated for 30 minutes at room temperature. Then, the plate was placed into BioTek Synergy H1 plate reader for read and continuous orbital rotation (180 cpm). Read speed was set at normal with 2000 msec delay and 30 measurements/data point. ThioT Fluorescence was measured using filter set 1 (excitation 440, emission 485, optics: top, gain 100) at 30 minute intervals over 48 hours. Averaged intensity is plotted after subtraction of values for wells containing buffer only. A 0^{th} order polynomial fit was used to smooth the data by averaging 3 data point neighbours using the method of Savistsky and Golay in GraphPad Prism. Lag time was calculated as the time it takes to deviate 20% from the running mean. Initial slope, or the rate of ThioT fluorescence increase, was taken for 3 hours after the lag time or from the lag time until the end of the experiment (48 hours). If the fluorescence did not deviate from the running mean and there was no lag time, the slope was taken for the last 3 hours. Data points were performed in quadruplicates.

### ThioT Competition Assays

Competition assays used a fixed 1 μM concentration of aSyn fibrils and 3 μM of ThioT. The compound was diluted in 30 mM Tris-HCl, pH 7.4, 0.1% BSA and added to the reactions in various concentrations. Reactions were incubated at room temperature for 1.5 hours before quantifying bound compound as described above for saturation binding assay. Fluorescence was determined with a Biotek plate reader using a 440/30 excitation filter and a 485/20 emission filter. Data points were performed in triplicate. Nonspecific fluorescence was measured in parallel reactions containing ThioT plus each concentration of competitor but without fibrils, and these measurements were subtracted from the reactions with fibrils to yield fibril-specific fluorescence.

Characterization of aSyn Fibril Growth Products Via Negative Stain TEM

Negative staining of the aSyn fibrils after 24 hour incubation with or without 6 μM compounds 8 and 10 was performed by applying a given fibril preparation to ultrathin Carbon 300 mesh Gold grids (Catalog 01824 G, Ted Pella). The grids were negatively glow discharged (13 mA, 45 s) using GloQube glow discharge system (Model #025235 EMS). A 10 μl fibril sample at appropriate dilution was applied to the glow discharged grid for 5 minutes with the carbon side facing the sample drop. Post-sample incubation, the grid was washed (6 times) with 50 μl of H₂O, washed once with 50 μl of 0.75% uranyl formate and stained with 50 μl of 0.75% uranyl formate for 3 min. The grids were blotted using filter paper, leaving a small amount of stain to air dry on the grid surface. Grids were imaged on a JEOL 1400 TEM operating at 120 kV to visualize the negatively stained fibrils.

### Molecular Docking

Compound structures were downloaded as 3D SDF files (Chembridge, Vitas M Laboratory). Molecular blind docking studies were performed via Autodock Vina⁵¹ (Scripps) and viewed with Pymol (pymol.org). The structure of LBD-derived aSyn fibrils determined by single particle cryo-EM (PDB ID: 8a9I) were obtained from RCSB protein data bank (https://www.rcsb.org/) as a target protein for blind docking. The LBD-derived aSyn structure 8a9I was increased to 5 fibril units using RELION, and only the beta-sheet regions aa 31-100 was kept. Water molecules and nonpolar hydrogens were removed from all compounds and protein structures. A grid box with a dimension of 126×126×126 Å³ was applied to all aSyn structures, and center x=86.868, y=95.080, and z=97.452 were applied for 8a9I. Energy range was set to 4 and exhaustiveness was set to 8 (default values).

What is claimed is:

1. A composition comprising at least one alpha-Synuclein (aSyn) inhibiting agent, wherein the at least one aSyn inhibiting agent is a compound comprising a dimethyoxyphenyl piperazine group according to

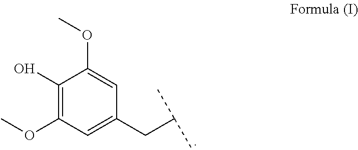

2. The composition of claim 1, wherein the compound comprising a dimethyoxyphenyl piperazine group is a compound according to

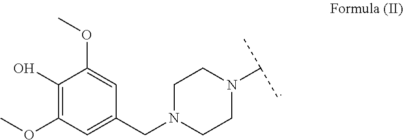

3. The composition of claim 2, wherein the at least one aSyn inhibiting agent is selected from Compound 5, Compound 8, Compound 9, Compound 10, Compound 11, Compound 12.

4. The composition of claim 2, wherein the at least one aSyn inhibiting agent is selected from Compound 13, Compound 14, Compound 15, Compound 16, Compound 17, Compound 18.

5. The composition of claim 2, wherein the at least one aSyn inhibiting agent is selected from Compound 19, Compound 20, Compound 21, Compound 22, Compound 23, and Compound 24.

6. A method of inhibiting alpha-synuclein (aSyn) fibril growth in a subject in need thereof, the method comprising:
administering to the subject a composition comprising at least one alpha-Synuclein (aSyn) inhibiting agent, wherein the at least one aSyn inhibiting agent is a compound comprising a dimethyoxyphenyl piperazine group according to

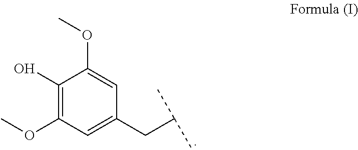

7. The method of claim 6, wherein the compound comprising a dimethyoxyphenyl piperazine group is a compound according to

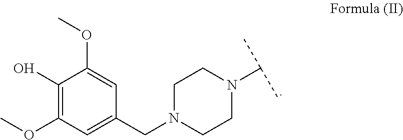

8. The method of claim 6, wherein the subject has Parkinson's Disease.

9. The method of claim 6, wherein the subject has Lewy Body disease (LBD).

10. The method of claim 6, wherein the inhibiting aSyn fibril growth further comprises preventing accumulation of aSyn fibrils in the subject.

11. The method of claim 7, wherein the at least one aSyn inhibiting agent is selected from Compound 5, Compound 8, Compound 9, Compound 10, Compound 11, Compound 12.

12. The method of claim 7, wherein the at least one aSyn inhibiting agent is selected from Compound 13, Compound 14, Compound 15, Compound 16, Compound 17, Compound 18.

13. The method of claim 7, wherein the at least one aSyn inhibiting agent is selected from Compound 19, Compound 20, Compound 21, Compound 22, Compound 23, and Compound 24.

14. A method of treating a neurodegenerative disease in a subject in need thereof, the method comprising:
administering to the subject a composition comprising at least one alpha-Synuclein (aSyn) inhibiting agent, wherein the at least one aSyn inhibiting agent is a compound comprising a dimethyoxyphenyl piperazine group according to

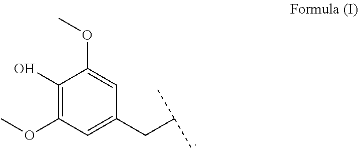

15. The method of claim 14, wherein the compound comprising a dimethyoxyphenyl piperazine group is a compound according to

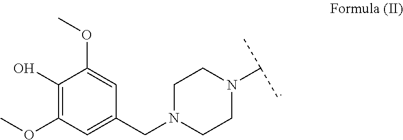

16. The method of claim 14, wherein the neurodegenerative disease is Parkinson's Disease.

17. The method of claim 14, wherein the neurodegenerative disease is Lewy Body disease (LBD).

18. The method of claim 15, wherein the at least one aSyn inhibiting agent is selected from Compound 5, Compound 8, Compound 9, Compound 10, Compound 11, Compound 12.

19. The method of claim 15, wherein the at least one aSyn inhibiting agent is selected from Compound 13, Compound 14, Compound 15, Compound 16, Compound 17, Compound 18.

20. The method of claim 15, wherein the at least one aSyn inhibiting agent is selected from Compound 19, Compound 20, Compound 21, Compound 22, Compound 23, and Compound 24.
